(19) United States
(12) Patent Application Publication
(10) Pub. No.: US 2004/0106128 A1
(43) Pub. Date: Jun. 3, 2004

(54) Cancer vaccines containing xenogeneic epitopes of telomerase reverse transcriptase

(76) Inventors: Anish Sen Majumdar, Sunnyvale, CA (US); Iris A. Ferber, San Jose, CA (US); Maria Frolkis, San Carlos, CA (US); Zhuo Wang, Fremont, CA (US)

Correspondence Address: GERON CORPORATION, 230 CONSTITUTION DRIVE, MENLO PARK, CA, 94025

(21) Appl. No.: 10/602,441

(22) Filed: Jun. 24, 2003

Related U.S. Application Data
(60) Provisional application No. 60/393,295, filed on Jun. 27, 2002.

(51) Int. Cl. C12Q 1/68

(52) U.S. Cl. 435/6

(57) ABSTRACT

It has been discovered that a robust and therapeutic anti-cancer response can be generated by immunizing with a xenogeneic form of the enzyme telomerase reverse transcriptase (TERT). Cancer subjects are multiply immunized with TERT from another species—either in protein form, or with a TERT expression vector. Presence of the xenogeneic components apparently overcomes natural immunotolerance to self-antigen. The response can be focused by simultaneous or subsequent immunization with isogenic TERT. As a result, the immune system generates T lymphocytes that are cytotoxic for virtually any cancer cell, by virtue of TERT expressed due to malignant transformation. The anti-tumor response causes a substantial inhibition of tumor cell growth, demonstrating the therapeutic benefit of this invention.

### REFERENCE TO RELATED APPLICATIONS

This disclosure claims the priority benefit of U.S. provisional application 60/393,295, filed Jun. 27, 2002. The priority application is hereby incorporated herein by reference in its entirety.

### TECHNICAL FIELD

This invention relates to the field of immunization strategies for the treatment of cancer. The invention overcomes tolerance to telomerase reverse transcriptase, a self-antigen expressed by cancer cells, generating a therapeutically beneficial anti-telomerase specific immune response.

### BACKGROUND

Telomere dynamics and telomerase expression are fundamentally involved in cellular aging and cancer. Activation of the genes for the telomerase complex is associated with cell immortality and human malignancies. Kim et al. (Science 266:2011, 1994) described the specific association of human telomerase activity with cancer cells. It has been proposed that cell immortalization is required for long-term growth of the vast majority of malignant or metastatic tumors, and that advances in telomere biology and telomerase inhibition will improve the way cancers are diagnosed and treated (Harley et al., Important Adv. Oncol. 57, 1996).

U.S. Pat. No. 5,583,016 describes the cloning and characterization of the RNA component of human telomerase. U.S. Pat. No. 6,300,110 describes the cloning and characterization of TPC2 and TPC3, two proteins that share with telomerase the property that expression levels increase in cancer cells. U.S. Pat. Nos. 6,517,834 and 6,545,133 describe the isolation of the human telomerase holoenzyme by affinity techniques. U.S. Pat. Nos. 6,166,178, and 6,261,836, and Nakamura et al. (Science 277:955, 1997) describe the cloning and characterization of the telomerase catalytic subunit, and its use to make anti-telomerase antibody.

U.S. Pat. No. 6,440,735 describes dendritic cell vaccines containing telomerase reverse transcriptase (TERT) for the treatment of cancer. The antigen-presenting cells contain or are transfected to express a portion of TERT. When the cellular composition is administered to human subjects such as cancer patients, it induces an anti-TERT immunological response. Dendritic cells primed in this way can also be used to stimulate cytotoxic T cells that are specific for TERT. As of the time of the filing of this application, a vaccine comprising dendritic cells pulsed with TERT mRNA was in Phase 1 clinical trials at the Duke University Medical Center. The data suggest the vaccine was very well tolerated, and resulted in the generation of an anti-telomerase immune response in almost all patients (Geron Press Release, Apr. 7, 2003).

These results are encouraging. However, it is not always convenient to prepare a dendritic cell composition from patients diagnosed with cancer. The present invention provides an alternative strategy for overcoming self-tolerance, thereby eliciting an anti-telomerase cytotoxic T cell response, which then helps eradicate the tumor.

### SUMMARY

This invention provides a system for eliciting an immune response in a mammalian subject that is specific for its own telomerase reverse transcriptase (TERT). In a typical embodiment, the subject is administered with an immunogenic composition containing part of a homolog TERT, either in protein form or encoded in a nucleic acid. In some instances, one or more homologs are administered in combination with simultaneous or sequential administration of isogenic TERT.

Ideal homologs are full-length TERT and TERT fragments of various lengths of species xenogeneic to the subject being treated—particularly mammals and other eukaryotes. Also suitable are naturally occurring TERT sequences modified with one or more amino acid changes introduced for any reason, such as to eliminate telomerase activity. Also suitable are artificial sequences that mimic TERT epitopes, such as optimized and consensus sequences.

Medicinal compositions of this invention can be formulated for clinical treatment of humans or other animals, or for research purposes. The TERT compositions of this invention are often administered to the subject several times: first to initiate the response, and then to potentiate or focus the effect. Desirable outcomes are to elicit or enhance an immunological response (such as a cytotoxic T cell response) against TERT or against the subject's cancer, and production of mediators like IL-8 that directly or indirectly influence cancer cell apoptosis or elimination. These events alone or in combination can result in modulation of tumor growth, and stabilization or improvement of the clinical condition.

Embodiments of this system include therapeutic compositions and combinations (packaged or distributed separately or together), methods for producing and testing such compositions, and the use of such compositions for preparing medicine and treating telomerase-associated disease.

Other aspects of the invention relate to novel and modified forms of TERT and the genes that encode them. This disclosure provides the first sequence data for dog TERT, and for consensus and variant forms of TERT. These embodiments have utility for research, diagnostic, and therapeutic applications, as exemplified below.

Additional aspects of the invention will be apparent from the description that follows.

### DRAWINGS

FIG. 1 shows that vaccination of tumor-bearing mice with dendritic cells (DCs) primed with adenovirus expression vector for human telomerase reverse transcriptase (hTERT) is more effective than mouse TERT (mTERT) in halting tumor growth.

FIG. 2 shows that tumor rejection in these mice correlates with the presence of IFN-γ producing T lymphocytes that are specific for TERT.

FIG. 3 shows the results of experiments in which animals were immunized not with dendritic cells, but with TERT expression vectors. T lymphocytes from individual mice immunized with hTERT are cytotoxic for B16F10 mouse melanoma tumor cells (transduced with AdhTERT or unmodified), for B10.2 mouse tumor cells, and for C57 mouse fibrosarcoma cells. The target antigen meditating cytotoxicity is thought to be mTERT expressed endogenously by the mouse tumors.

FIG. 4 shows IFN-γ expression by T cells from two experiments in which mice were vaccinated according to the protocol shown on the abscissa. Specific CD positive cells predominate in mice immunized with xenogeneic hTERT.

FIG. 5 shows that xenogeneic antigen followed by self antigen is better than self antigen alone in generating specific cytotoxic T lymphocytes, and lymphocytes that specifically produce IFN-γ.

FIG. 6 is taken from an experiment in which mice were immunized four times with mTERT DNA, or twice each with hTERT DNA followed by mTERT DNA. The mice immunized with a combination of hTERT and mTERT had more mTERT-specific CTLs. The level of killing is higher in this experiment because the target cells had been transduced with AdmTERT. This confirms that the killing of the tumor cells is mediated by the TERT antigen.

FIG. 7 shows that immunizing with a combination of hTERT and mTERT leads to a specific CTL response against autologous antigen. The mice were immunized nine times with 100 μg DNA followed by electroporation. Splenocytes were harvested and stimulated with irradiated mTERT expressing cells. mTERT-specific killing (mean±SEM) was highest for animals multiply immunized with xenogeneic TERT, followed by isogenic TERT.

FIG. 8 shows that immunization with xenogeneic TERT effectively inhibits tumor growth. Mice were immunized three times with adenovirus hTERT virus expression vector or control vector. The animals immunized with xenogeneic hTERT (diamonds) resisted tumor growth by almost 3-fold, compared with vector control (p<0.05).

FIG. 9 is an alignment of TERT protein sequences from human (SEQ. ID NO:2), mouse (SEQ. ID NO:4), hamster (SEQ. ID NO:6), rat (SEQ. ID NO:8), and dog (SEQ. ID NO:10). Shown below is a consensus sequence (SEQ. ID NO:12).

FIG. 10 is an alignment of TERT encoding gene sequences from human (SEQ. ID NO:1), mouse (SEQ. ID NO:3), hamster (SEQ. ID NO:5), rat (SEQ. ID NO:7), and dog (SEQ. ID NO:9). Shown below is a consensus sequence (SEQ. ID NO:11).

### DETAILED DESCRIPTION

Normally, the telomeres that form the aglet on the ends of chromosomes shorten a small amount after each cell division. This limits the replicative capacity of mammalian cells to 50-100 divisions, before they undergo replicative senescence.

Telomerase serves a key role in preventing replicative senescence in immortal cell lines by periodically restoring length to the telomeres. It is expressed by embryonic stem cells, which can be grown in culture indefinitely (WO 01/51616). It is also expressed in a transient fashion in adult cells with special replicative requirements, such as certain tissue-specific stem cells, and T lymphocytes during activation. However, most adult cells don't express telomerase reverse transcriptase (TERT, the catalytic component of the enzyme) unless they undergo malignant transformation.

The expression pattern of TERT makes it a good target for a cancer vaccine. However, since telomerase is a self-antigen, there is a formidable problem in overcoming the natural process of immune tolerance to self-antigens in order to summon an adequate immune response to have a serious impact on a growing tumor.

This disclosure overcomes the problem by demonstrating that cross-species epitopes can be used as a way of initiating an effective anti-TERT response for the treatment of cancer.

The discovery was made in part during the course of experiments in which mice were injected with expression vectors for autologous TERT. As a positive control, cohort mice were immunized with human TERT, which is over 30% different from mouse TERT and therefore should be quite immunogenic. When T cells were isolated form these mice, it was found that they could kill target cells transfected to express human TERT, as expected. Surprisingly, they were also found to kill target cells expressing mouse TERT—both in transfected cells, and from the endogenous mTERT gene expressed in mouse tumor cells. This demonstrates that immunization with cross-species epitopes overcomes tolerance to self-antigen, and elicits a response sufficiently cross-reactive that it mediates killing of tumor cells of the host species.

As a refinement to this technique, cross-species TERT epitopes can be used in conjunction with autologous TERT epitopes. The subject is immunized with cross-species epitopes to overcome self-tolerance and begin the generation of cross-reactive T cells specific for autologous TERT. Simultaneously or at a later time, the subject is also immunized with autologous TERT, which serves the function of focusing the response towards autologous epitopes, promoting maturation of the immune response in the direction of high-affinity reactivity against antigen expressed on tumor cells.

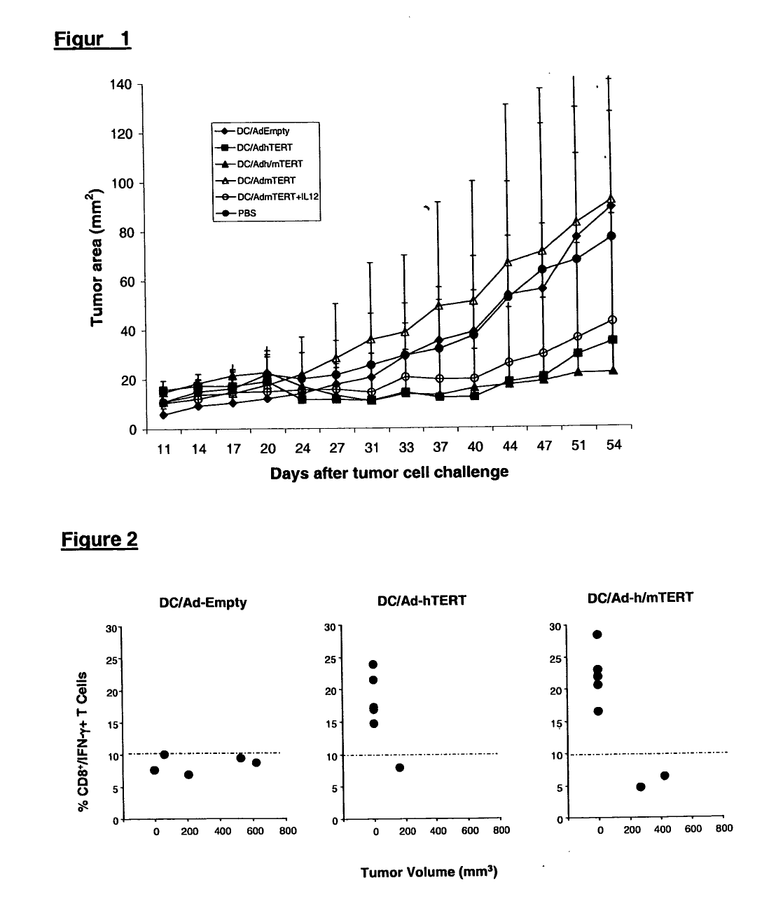

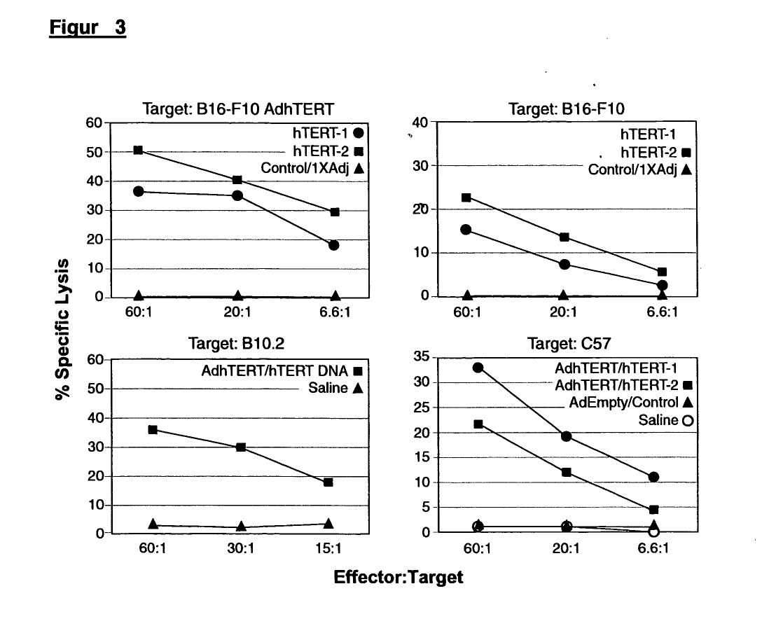

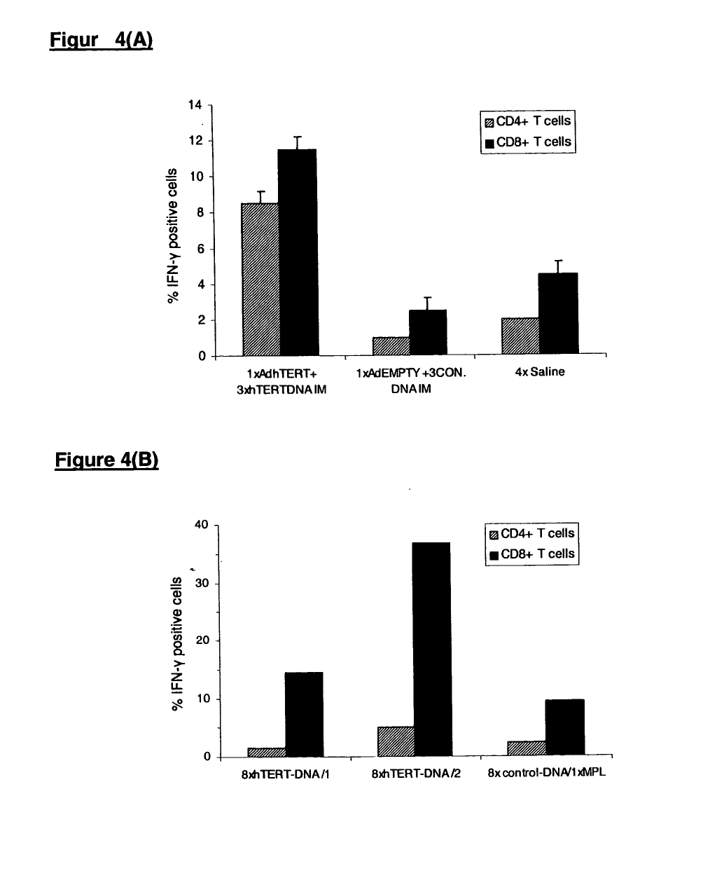

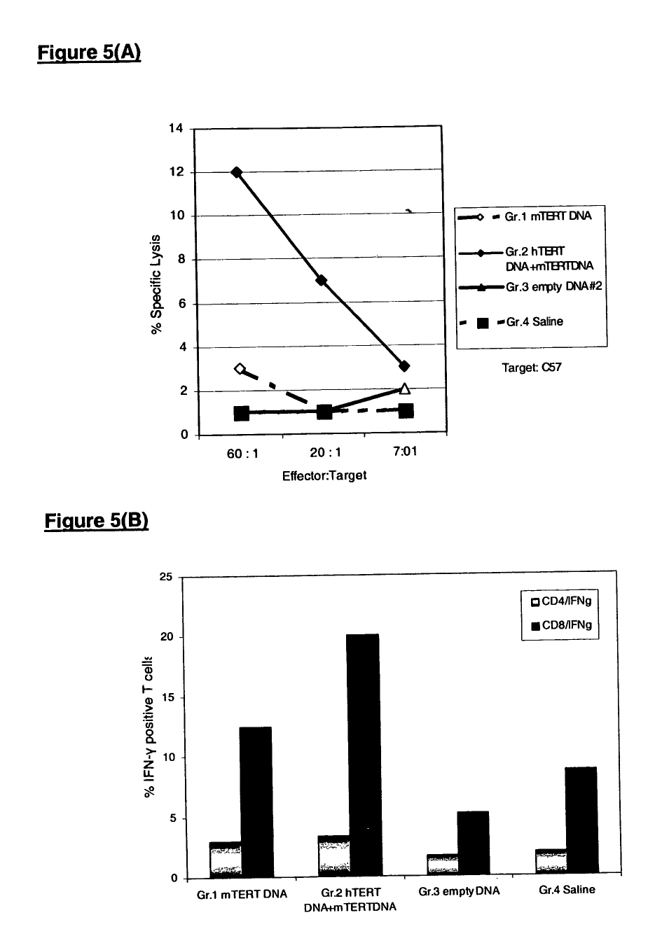

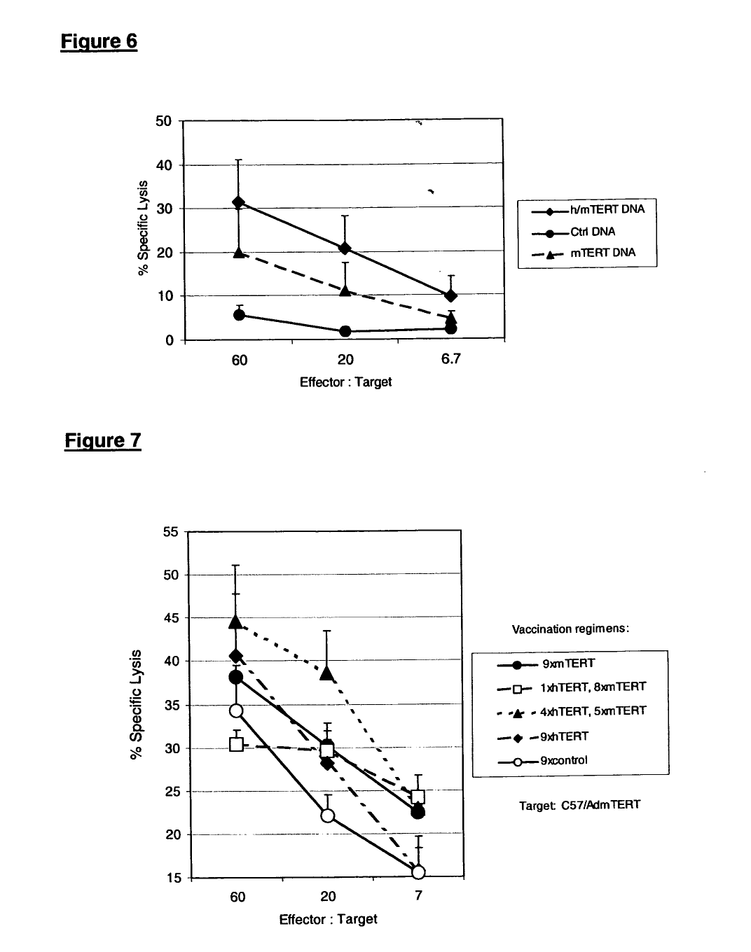

Use of this strategy to overcome tolerance and raise a response against syngeneic tumor cells is illustrated in the Example section appearing later in this disclosure. FIG. 1 shows that mice injected with dendritic cells expressing human TERT (or hTERT followed by mouse TERT) are better protected against tumor challenge than mice injected with mTERT alone. As it turns out, using cross-species epitopes to elicit a protective response does not require the presence of dendritic cells in the composition. FIG. 3 shows CTL activity against a variety of different mouse tumor cells in T cells obtained from mice immunized with hTERT. FIG. 4 shows that T cells with the CD8 (CTL) phenotype that express IFN-γ are specifically elicited in the mice immunized with hTERT. FIGS. 5 and 6 show that the combination of human and mouse TERT vaccination using a DNA expression vector is better than vaccination with mTERT DNA alone, and that the proportion of target cell killing depends on the amount of mTERT they express—confirming that mTERT is the antigen being recognized.

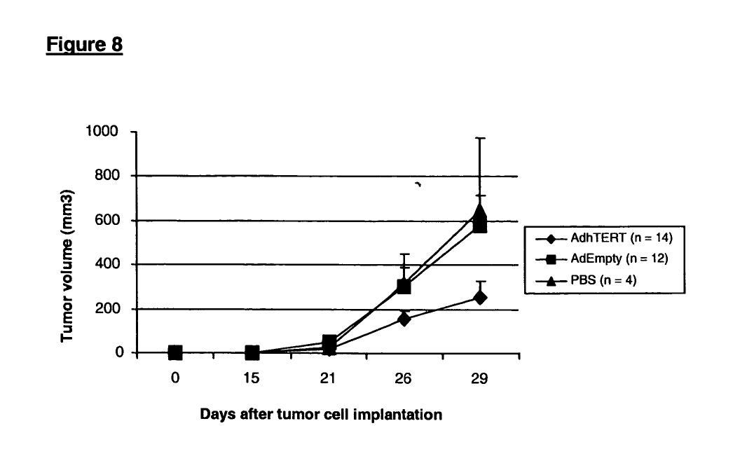

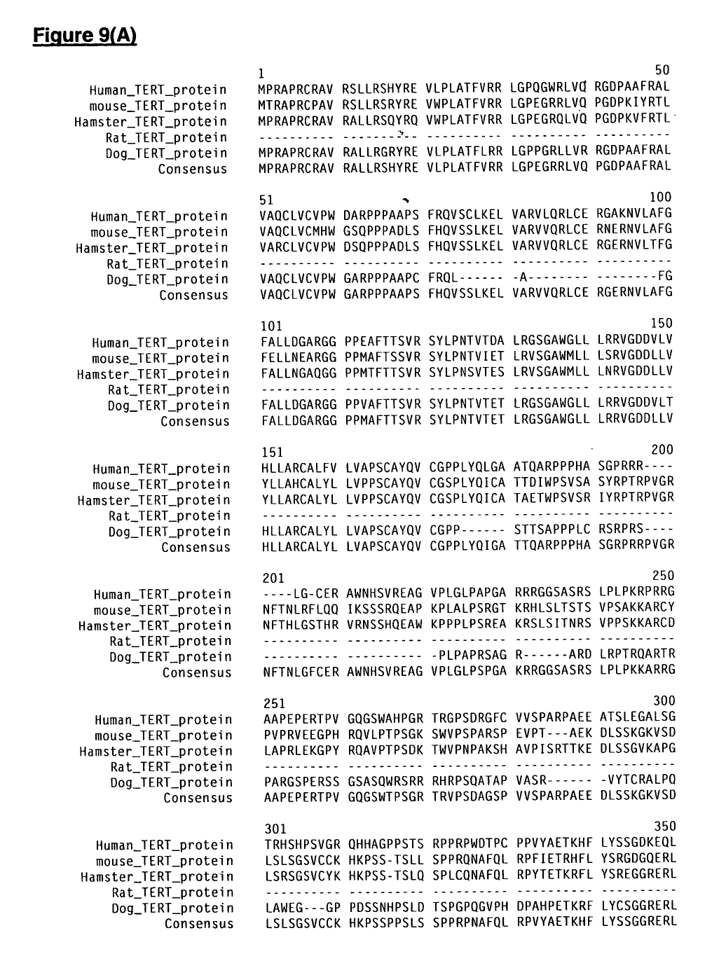

FIG. 7 shows that the strategy of immunizing first with cross-species TERT, and then with autologous TERT, provides higher levels of CTL killing than immunizing with either TERT alone. FIG. 8 demonstrates that the CTL response elicited by cross-species TERT is protective against syngeneic tumor cells.

Accordingly, it is now possible with an acellular vaccine composition comprising cross-species epitopes to generate a CTL response that provides a therapeutic benefit against cancer.

The strategy illustrated in these examples is readily adapted to human therapy by using a non-human TERT or portion thereof to provide the cross-reactive epitopes that overcome self-tolerance and initiate a response that cross-reacts with autologous TERT. Optionally, the patient is also treated with human TERT or a portion thereof to focus the response through affinity maturation towards the intended target on the tumor cells. Indeed, the mouse is a more rigorous test of the viability of this strategy because unlike in humans, TERT is endogenously expressed by most adult mouse cells. Thus, self-tolerance against TERT epitopes will be promoted more vigorously in the mouse on an ongoing basis. Adapting the strategy to human therapy brings it into a less tolerized host, generating CTLs against autologous TERT in a less stringent system.

General Techniques

For further elaboration of general techniques useful in the practice of this invention, the practitioner can refer to standard textbooks and reviews in cell and molecular biology, tissue culture, and veterinary and human medicine.

Reference books for molecular genetics and genetic engineering include the current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.).

The association of telomerase expression with cancer is reviewed by Harley & Kim (Important Adv. Oncol. 57-67, 1996). General information on telomerase, telomere biology, and related techniques is provided in Telomerases, Telomeres and Cancer (G. Krupp & R. Parwaresch eds., Plenum Pub. Corp. 2002); Telomeres and Telomerase: Methods and Protocols (J. A. Double & M. J. Thompson eds., Humana Press 2002); and Telomerase, Aging and Disease (M. P. Mattson ed., Elsevier Science 2001).

Cross-Species Telomerase Epitopes

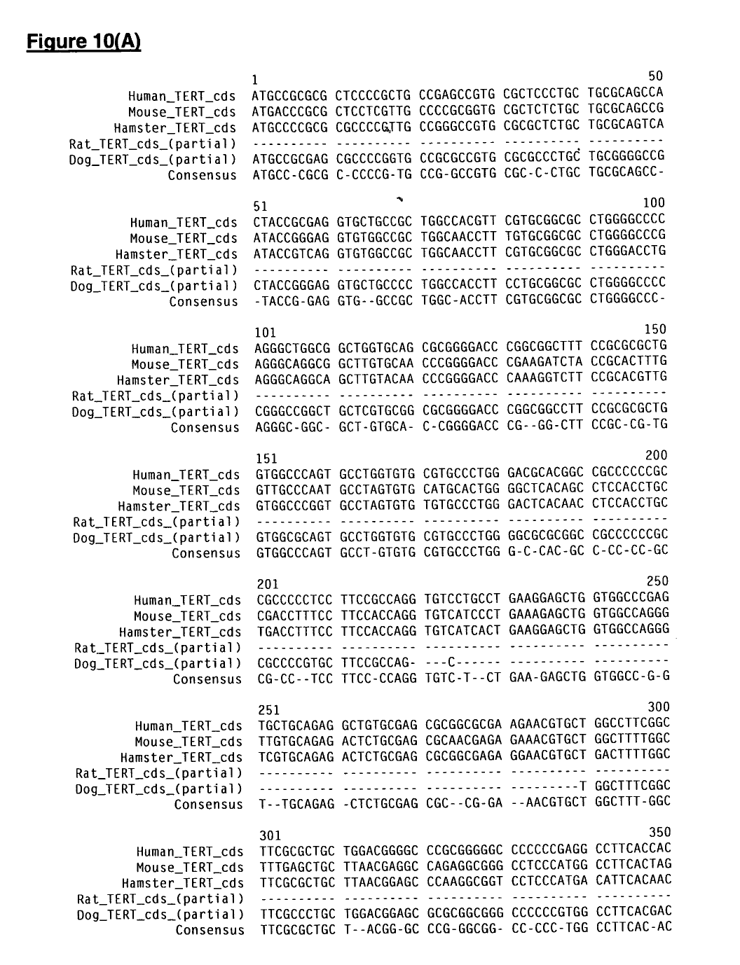

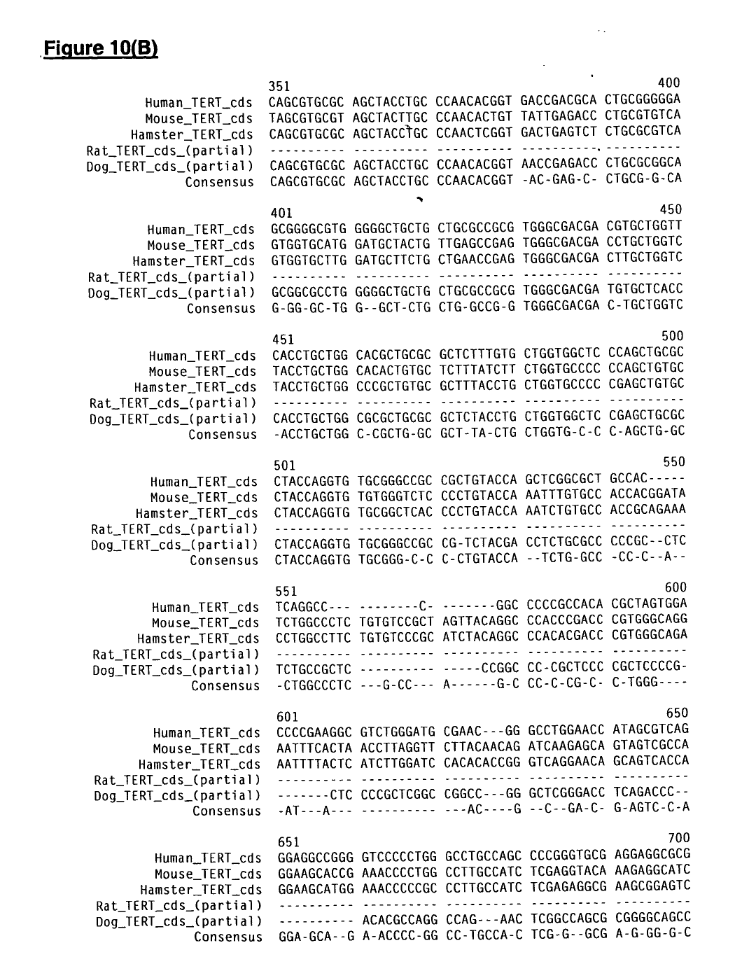

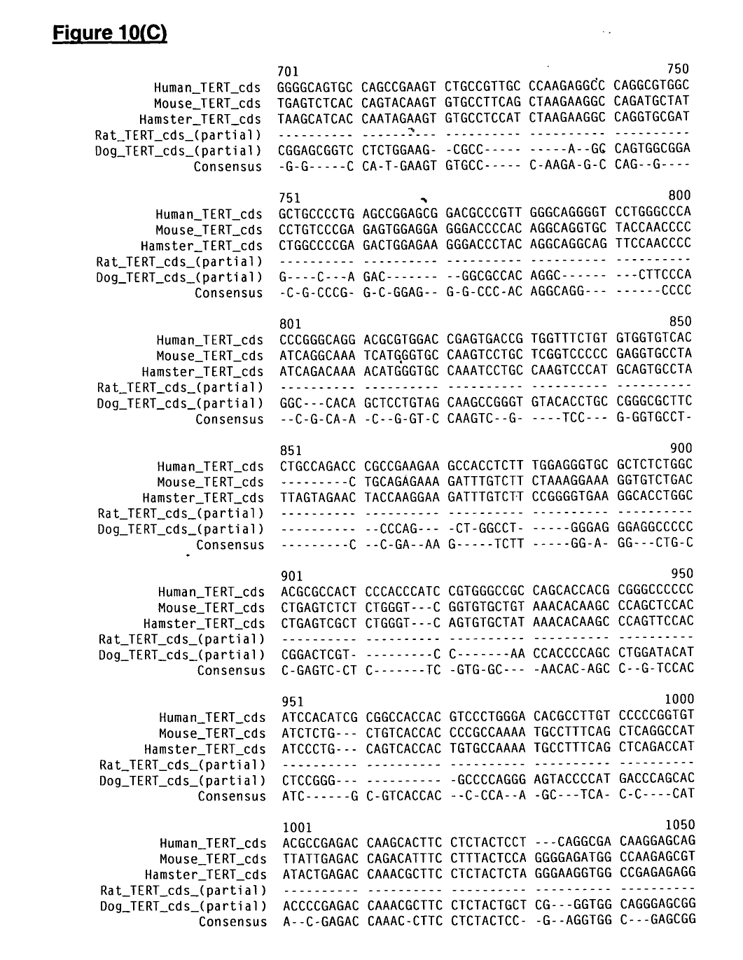

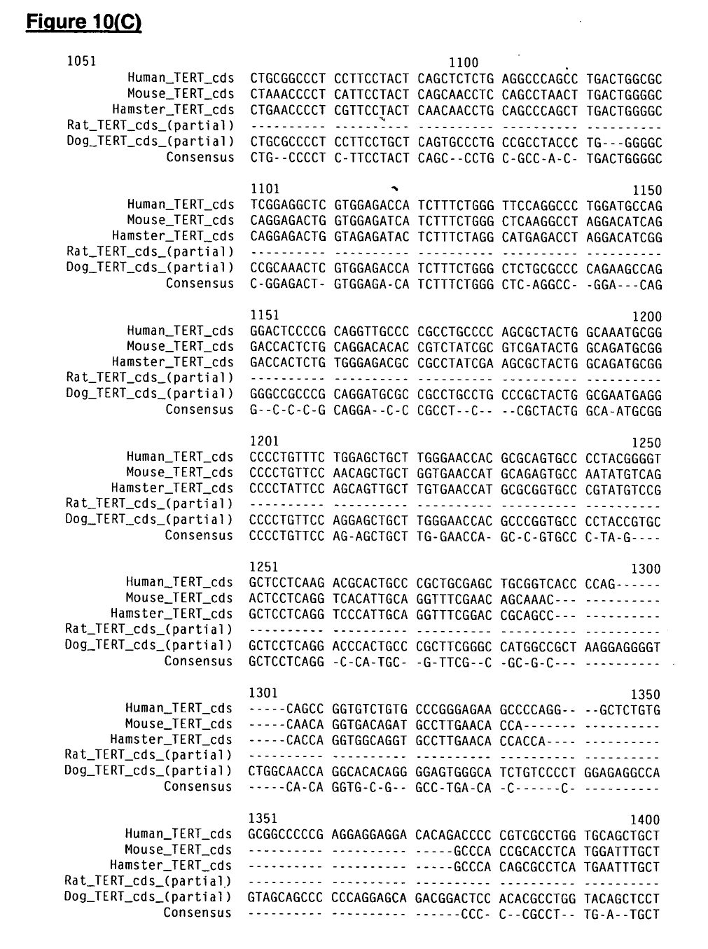

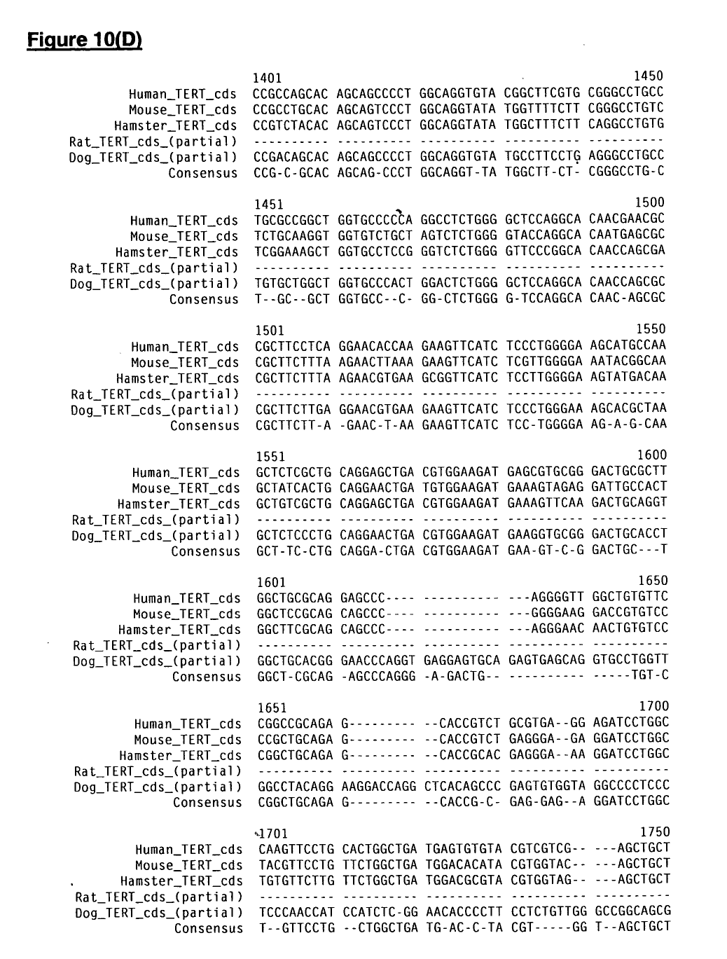

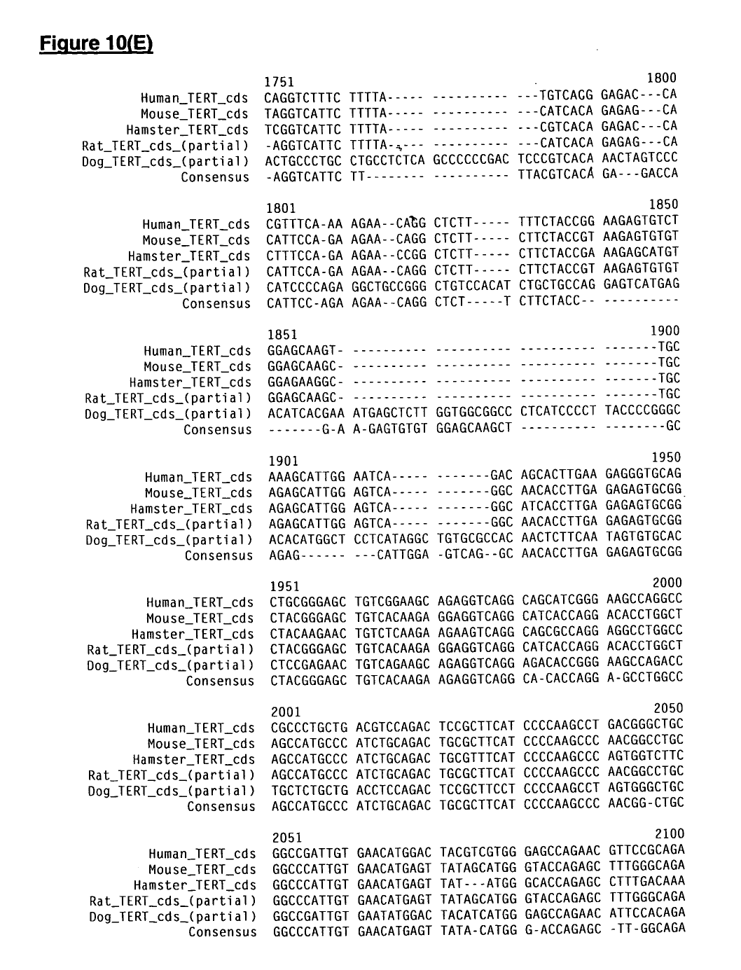

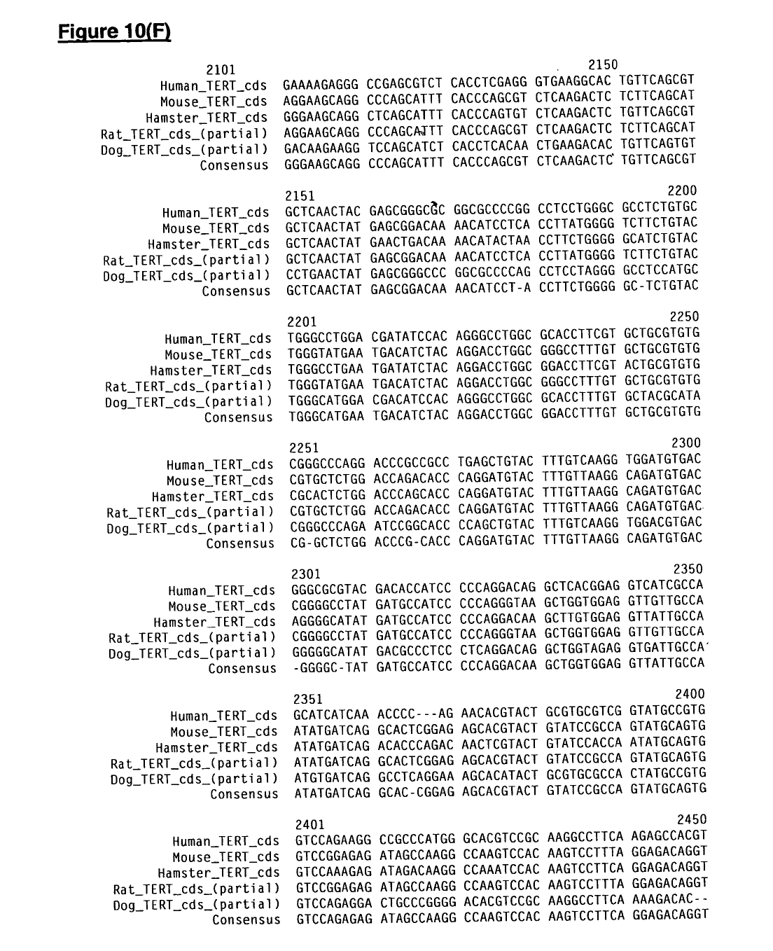

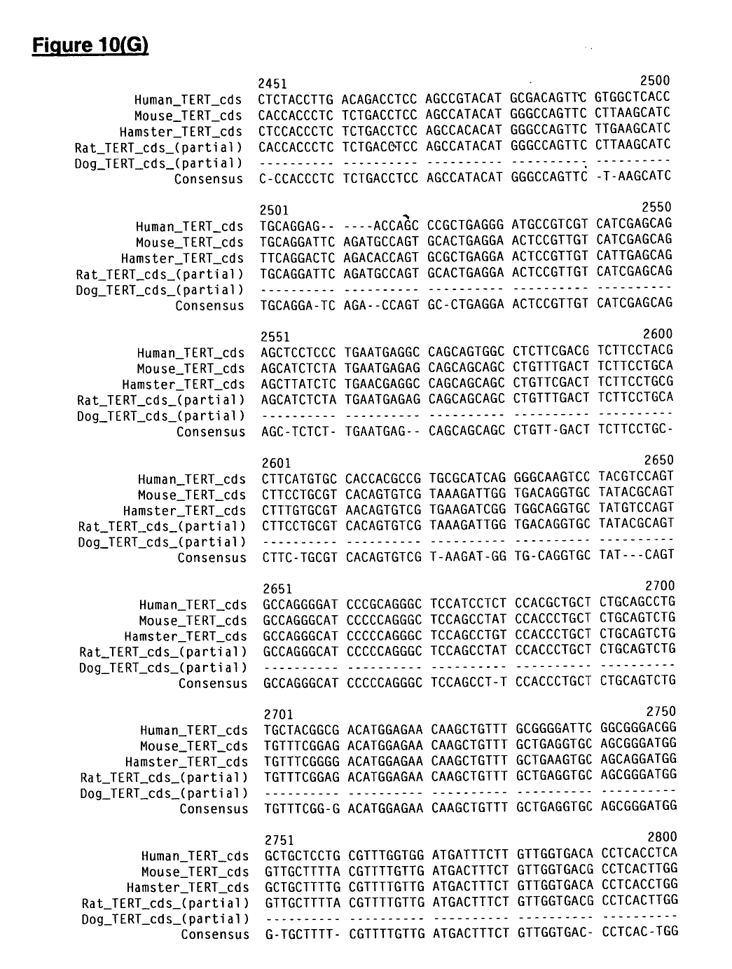

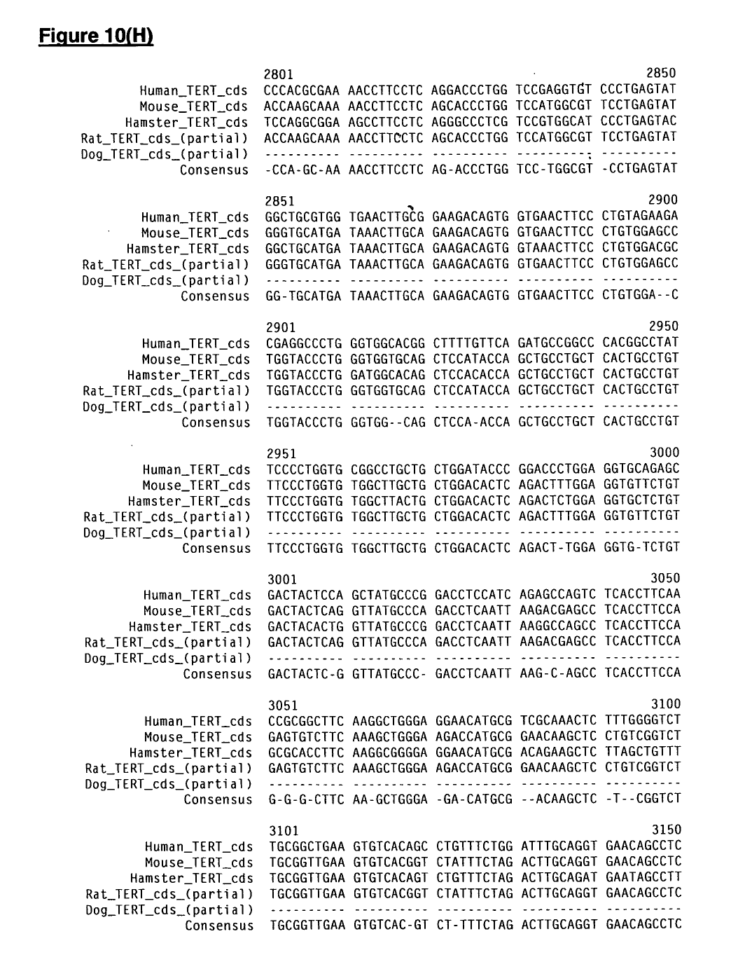

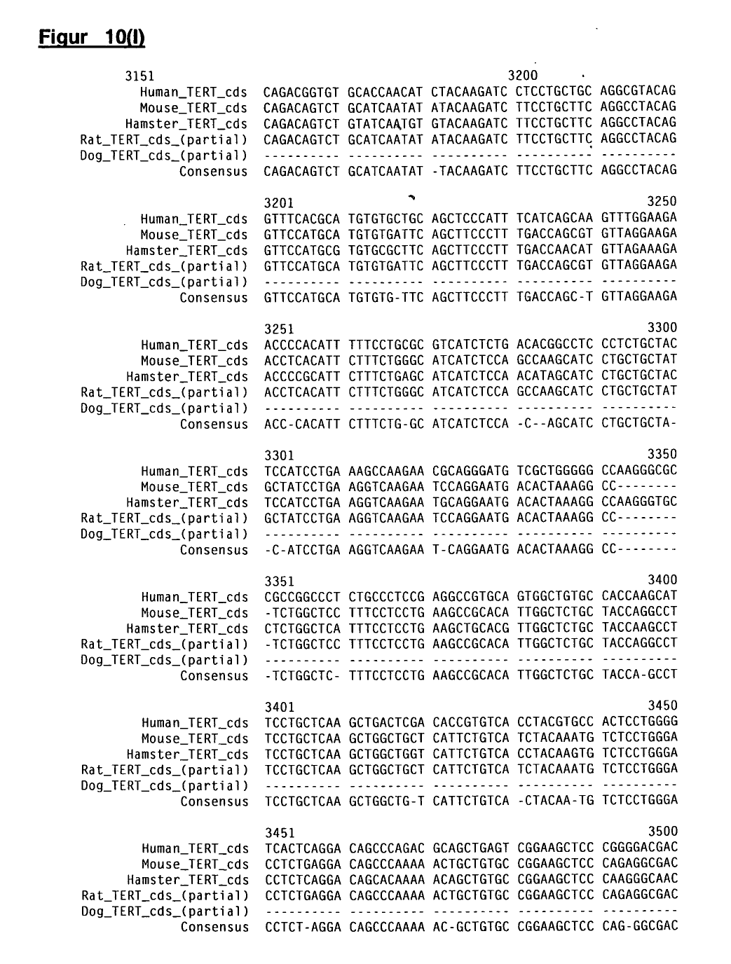

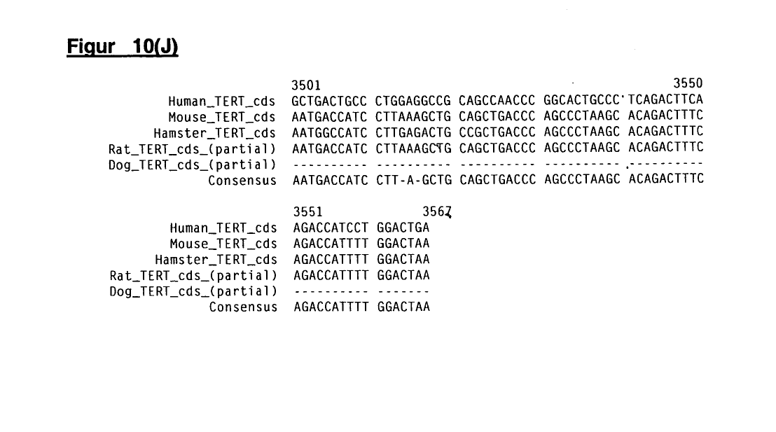

The source of cross-species TERT epitopes can be any species other than the one being immunized. Exemplary are human TERT (SEQ. ID NOs:1 and 2) in the mouse, and mouse TERT (SEQ. ID NOs:3 and 4) in the human. Other TERT species are also suitable for treating mammals, particularly if they are from another vertebrate or mammal. Further TERT species are listed in SEQ. ID NOs:5 to 10. Also suitable are consensus sequences, designed by compiling sequence information from other vertebrates, as exemplified in FIG. 10.

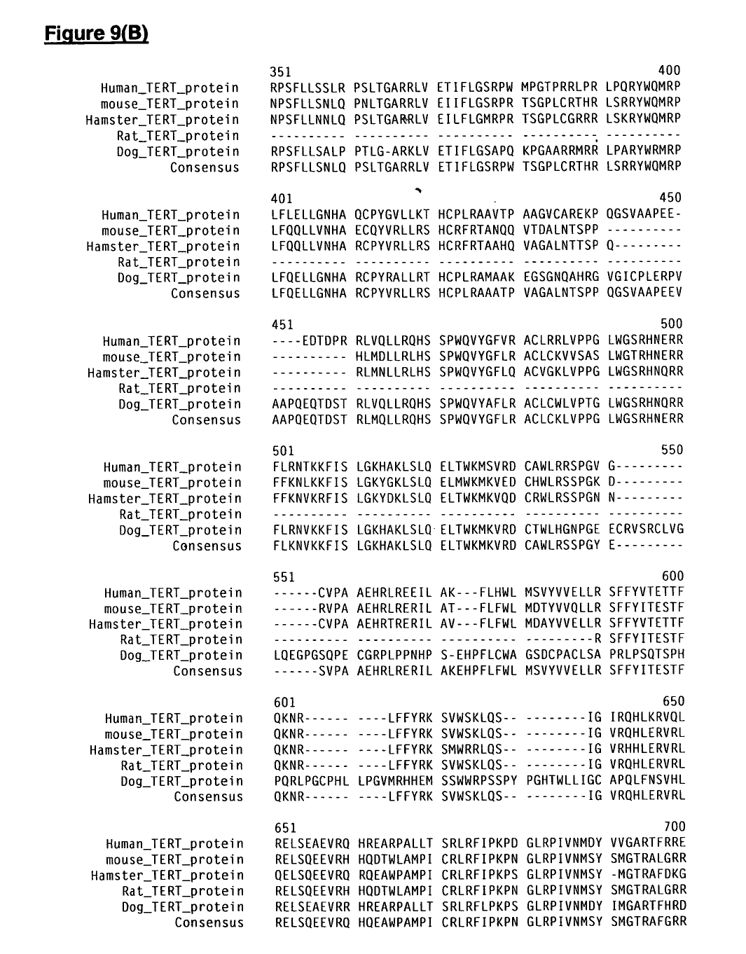

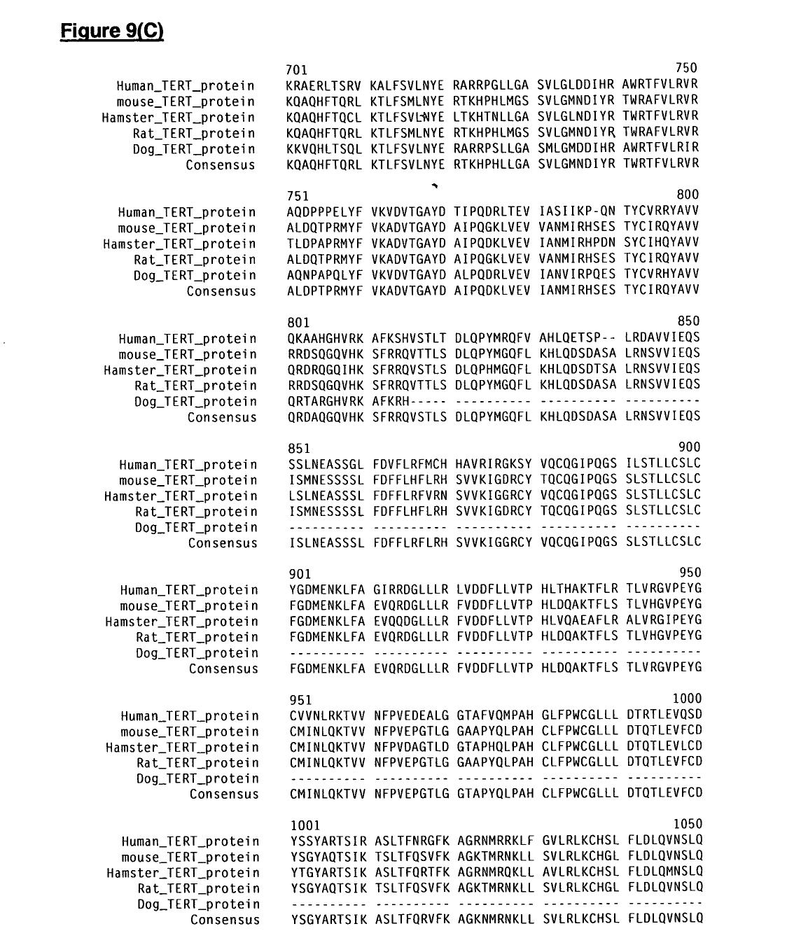

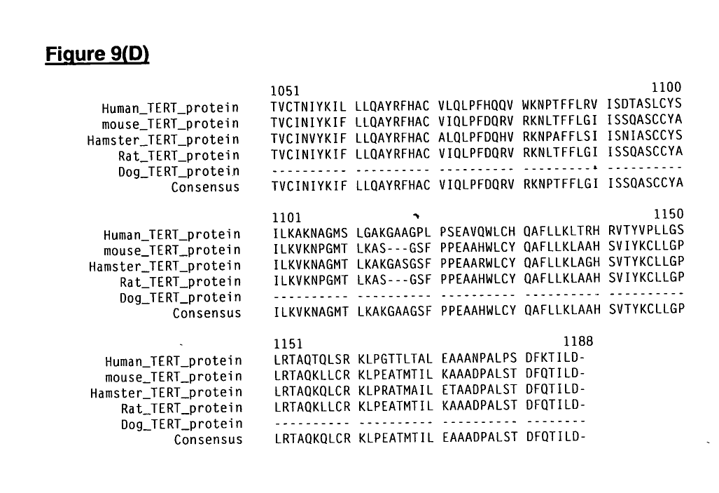

Other artificial sequences based on mammalian TERTs can also be used, in order to overcome self-tolerance and prime the cross-reactive response to autologous TERT. Such artificial sequences will typically share sequence identity across shared residues amongst the TERT family (FIG. 9), and one or more of the telomerase motifs described in U.S. Pat. No. 6,166,178, Nakamura et al. (Science 277:955, 1997), or Bryan et al. (Proc. Natl. Acad. Sci. USA;95:8479, 1998). Characteristic telomerase motifs have the following structures:

| | |
|---|---|
| Motif T | W-R¹-X₇-R¹-R¹-R²-X-F-F-Y-X-T-E-X₈₋₉-R³-R³-R-R⁴-X₂-W |
| Motif 1 | X₃-R-X₂-P-K-X₃ |
| Motif 2 | X-R-X-I-X |
| Motif A | X₄-F-X₃-D-X₄-Y-D-X₂ |
| Motif B′ | Y-X₄-G-X₂-Q-G-X₃-S-X₈ |
| Motif C | X₆-D-D-X-L-X₃ |

where R′ is Leu or Ile; R² is Gln or Arg; R³ is Phe or Tyr; R⁴ is Lys or His, and Xₙ represents the number n of consecutive unspecified amino acids. Other naturally occurring TERTs from additional species can be obtained either by identifying ESTs in expression libraries according to the telomerase motifs, or by cloning them from mRNA libraries using suitable primers based on encoding regions conserved amongst TERT species (FIG. 10). Effectiveness of TERT homologs is best determined empirically, as illustrated in the Example section below.

A vaccine composition of this invention can be provided in the form of intact TERT protein or TERT fragments comprising at least one immunogenic epitope, typically in the range of 10, 20, 50, 100, 200, 500, or 1000 consecutive amino acids of the full-length sequence. Unless explicitly stated otherwise, reference in this disclosure to TERT protein, TERT peptide or TERT fragment refers interchangeably to portions of naturally occurring telomerase reverse transcriptase of any length. The peptide can be produced by artificial peptide synthesis, recombinant expression, or purification from natural sources.

Combinations of full length TERT or TERT fragments from 2, 3, or more different species are also contemplated, wherein one of the species is optionally the same as the species being treated, and the others provide a combination of xenogeneic epitopes from different sources. The TERT proteins may be combined in the same composition, or prepared as separate medicaments for immunization of the subject at the same or different times.

For use in immunogenic compositions, the TERT protein may or may not have telomerase activity when associated with telomerase RNA component. If telomerase activity is present, then it may impart increased telomerase activity near the injection site, as can be determined by TRAP assay (Kim et al., Science 266:2011, 1997).

It is often desirable to prevent telomerization of the injection site, by using a telomerase protein that is functionally inactive. This can be accomplished by using a single peptide or combination of separate peptides, none of which is of adequate length to retain telomerase activity. In a preferred embodiment, the composition comprises a mixture of overlapping or non-overlapping peptides of between about 10-50 (say, about 20-25) consecutive amino acids, spanning some or all of the full length of the naturally occurring TERT. In choosing peptides or TERT regions to be included in the composition, it is beneficial to select those parts of the molecule containing one or more T cell epitopes. Immunogenic epitopes in human TERT are known (Lev et al., Cancer Res. 62:3184, 2002). Databases and algorithms for identifying other T cell epitopes are available (Rammensee et al., Immunogenetics 50:213, 1999; Schirle et al., J. Immunol. Methods 257:1, 2001; Lu et al., Cancer Res. 60:5223, 2000).

Another way to produce a composition devoid of telomerase activity is to adapt the TERT by amino acid mutation or deletion to eliminate telomerase activity. Mutations in the motifs indicated above, such as removing or replacing the Asp residues in the A, B, or C motif, may reduce or abolish telomerase activity (U.S. Pat. No. 6,166,178). See also U.S. Pat. No. 6,337,200 for a description of suitable adaptations that eliminate telomerase activity while preserving useful epitopes. Adaptations effective in removing enzyme activity from the TERT gene of one species can usually be adapted in species orthologs or artificial homologs by making the same change at the corresponding position, determined by motif analysis or alignment of the two sequences.

Alternatively or in addition to TERT protein, the vaccine can contain a polynucleotide designed to cause the expression of TERT peptide after administration to the host. The encoded peptide can constitute any of the TERT orthologs, homologs, or fragments already described in any effective combination. The encoding region for the protein will typically be situated in the polynucleotide under control of a suitable tissue-specific or endogenous promoter. Suitable vector systems include naked DNA plasmids, liposomal compositions to enhance delivery, and viral vectors that cause transient expression. Exemplary are adenovirus vectors and vectors of the herpes family, especially in a non-replicative form.

This disclosure also provides new sequence data for Canis familiaris (dog) TERT, and for consensus and variant forms of TERT. The protein sequence and protein-encoding nucleotide sequences of the TERT family (either full-length, or in fragment form as already discussed) have many important applications. For example, they can be used for eliciting an immune response, increasing cell proliferation, determining TERT expression in cells and tissues, clinical diagnosis, and identification of telomerase inhibitors (U.S. Pat. Nos. 6,166,178, 6,261,836, 6,440,735, 6,444,650, and 6,475,789; PCT publications WO 99/27113 and WO 02/91999). Gene sequence upstream from the encoding region contains the TERT promoter, which also has several important applications, such as promoter-reporter, constructs, TERT-targeted vectors and oncolytic virus, and elimination of stem cells with undifferentiated phenotype (U.K. Patent GB 2321642; PCT publications WO 00/46355; WO 02/42468; and WO 02/42445).

This invention includes amongst its embodiments all of these applications of TERT, adapting the descriptions of the aforelisted disclosures mutatis mutandis with the novel sequence information listed herein. For the purpose of prosecution and interpretation of this disclosure in the U.S., the aforelisted patent publications are hereby incorporated herein by reference in their entirety.

Formulation

Skilled readers will already appreciate that telomerase protein and nucleic acid intended for use in clinical therapy of human or animal subjects will typically be formulated as a medicament that is both compatible with the nature of the active ingredient, and with the subject being treated. Dry powders can be used in certain contexts, but the active ingredient is often provided in the presence of a pharmaceutically compatible excipient. The entire composition will be produced under appropriate conditions, rendered sufficiently sterile and free of undesired contaminants in a manner that makes it suitable for administration to the subjects intended for treatment.

Formulation of pharmaceutical compounds will accord with contemporary standards and techniques. Medicaments intended for human administration will be prepared in adequately sterile conditions, in which the active ingredient(s) are combined with an isotonic solution or other pharmaceutical carrier appropriate for the recommended therapeutic use. Suitable formulations and techniques are generally described in the latest edition of Remington's Pharmaceutical Sciences (Maack Publishing Co, Easton Pa.). With respect to the use of nucleic acid vectors in therapeutic applications, the reader may wish to consult The Skin and Gene Therapy (U. R. Hengge & B. Volc-Platzer eds., Springer Verlag, 2000), or Gene Therapy (Advances in Pharmacology, Vol 40) (J. T. August, J. Coyle & M. W. Anders eds., Academic Press 1997). Aspects of the preparation and use of vaccines for the treatment of cancer can be found in such standard reference books as Cancer Vaccines and Immunotherapy, P. L. Stern et al. eds., Cambridge University Press 2000; DNA Vaccines: Methods and Protocols, D. B. Lowrie & R. Whalen eds., Humana Press 1999; Peptide-Based Cancer Vaccines, W. M. Cast, Landes Bioscience 2000; Therapeutic Vaccination Strategies, H. Hennekes et al. eds., Springer Verlag 2000; New Vaccine Technologies, R. W. Ellis ed., Landes Bioscience 2001; and Vaccine Adjuvants, D. T. O'Hagan ed., Humana Press 2002.

If appropriate, the immunogenic compositions of this invention can be combined with an adjuvant to potentiate the immune response. Classic adjuvants include oil emulsions, like complete Freund's adjuvant, and adherent surfaces such as alum. Adjuvants that recruit dendritic cells or help elicit cytotoxic T cells are especially useful, since telomerase is an antigen that is internal to the cell, and not usually expressed externally except in the context of the MHC. Other factors that otherwise boost the immune response or promote apoptosis or elimination of cancer cells can also be included in the composition. As illustrated below, particular factors of interest include but are not limited to IL-12, GM-CSF, IL-2, and MPL.

Multiple doses or different combinations of the immunogenic compositions of this invention can be packaged for distribution separately or together. Each composition or set of compositions can be accompanied with written instructions (in the form of promotional material or a package insert) regarding the use of the composition or combination in eliciting an immune response or the treatment of cancer.

Use for Immunization and Cancer Treatment

The manner in which the immunogenic compositions of this invention are used will depend on their formulation and the needs of the subject to be treated. If the subject is adequately primed, then a single administration may be sufficient, but multiple administrations (say, at least 2 or 4) are more usual. Separate doses can be given on a weekly or biweekly schedule, or as needed, until an adequate response is obtained, or on an ongoing basis. Depending on empirical optimization for treatment of particular conditions, nucleic acid vectors and protein vaccines of xenogeneic and isogenic TERT can be used in any effective combination. Combinations of current interest include multiple priming doses of xenogeneic TERT (or mixed xenogeneic and isogenic TERT). Use of adenovirus vectors in the priming stage may add to immunogenicity. The subsequent boosting or focusing phase can comprise multiple administrations of TERT (possibly isogenic TERT) as a DNA plasmid or peptide fragments. Initial trials may be conducted with 6 to 10 sequential administrations, and adjusted according to the findings obtained.

Effective doses of vaccines may fall within the range of 10 to 500 μg of TERT protein, or 1 to 500 μg of nucleic acid, depending on size of the subject, activity of the promoter and other factors. Suitable subjects include mammals of any kind, including research animals, livestock, pets, and human or non-human primates. Included are subjects that have (or are suspected of having) cancer or any other TERT-associated condition, and unaffected controls. A list of cancers suitable for treatment may be found in U.S. Pat. No. 6,166,178.

Other agents can be included in the protocol as part of a combination treatment strategy. For example, Flt-3 Ligand (˜20 μg/kg) can be administered daily for a week or two in advance of each injection of TERT to mobilize dendritic cells and enhance the response (Evans et al., Vaccine 21:322, 2002; Disis et al., Blood 99:2845, 2002). IL-2 (Pomer et al., Urologe 34:215, 1995) and GM-CSF (Simmons et al., Prostate 39:291, 1999) are two examples of agents that can be injected systemically to potentiate the immunization effect. An agent that depletes regulatory T lymphocytes, such as ONTAK® (denileukin diftitox, a recombinant diphtheria toxin) can be administered to the subjects ˜4 days in advance of the first dose of the vaccine, or as appropriate to down-regulate T lymphocyte mediated suppression, and overcome tolerance to self-antigen.

Desirable outcomes of the use of the compositions and treatment methods of this invention include activation of the immune system, and (if the treated subject previously had a telomerase-associated disease) improvement in the subject's clinical status. From an immunological standpoint, a T cell response is especially desirable, which can be measured in a proliferation assay (e.g., ELISPOT assay; D. I. Stott, J. Immunoassay 21:273, 2000), a cytotoxicity test (FIG. 3), or a specific cytokine secretion assay (FIG. 4). To measure an anti-hTERT response, the target cells can be hTERT transduced cells. To measure an anti-tumor response, the target cells can be a cancer cell line of the same tumor type as the subject. A particularly sensitive test is to take PBMCs from the immunized subject, make dendritic cells from the adherent fraction by culturing with IL-4 and GM-CSF, and transfect the DCs to express isogenic TERT. An assay is then run to ask whether T cells in the non-adherent fraction will respond when the autologous TERT-presenting DCs are used as stimulator cells.

Clinical objectives include inhibition of tumor growth (measured by a suitable technique such as caliper calibration or MRI), tumor regression, improved survival rate, and improved quality of life. Ultimate choice of the treatment protocol, dose, and monitoring is the responsibility of the managing clinician.

The examples that follow are provided by way of further illustration, and are not meant to limit the claimed invention.

### EXAMPLES

Materials and Methods

hTERT and mTERT plasmid DNA vectors were used in all experiments. Full-length hTERT coding sequence was cloned into a high expression vector, gWiZ™ (Gene Therapy Systems, San Diego, Calif.) under control of a modified CMV promoter. A gWiZ™ mTERT vector was constructed by inserting the full-length mTERT gene in the same vector. gWiz™ blank vector was purchased from Genetic Therapy Systems and used as a negative control.

AdhTERT is a replication deficient, E1 and E3 deleted, recombinant adenovirus 5 based vector containing a cassette encoding the human telomerase gene under the control of CAG promoter construct (cytomegalovirus enhancer, chicken β-actin promoter, and part of the 3′ untranslated region of rabbit β-globin gene). The AdhTERT virus was generated by COS-TPC method (references 1 & 2) using the Adenovirus Expression Vector Kit from Takara Biomedicals (Tokyo, Japan). Briefly, the 3419 bp hTERT fragment was cloned into Swal site in pAxC wt cosmid (Takara Biomedicals, Tokyo, Japan) and transfected into 293 cells. The desired recombinant adenovirus was generated by homologous recombination in 293 cells. After several rounds of plaque purification, the rAdhTERT viruses were amplified in 293 cells and purified by CsCl density gradient ultracentrifugation. Viral particle concentration was determined by measuring optical density at A₂₆₀ and the infectious titer was determined by standard plaque assay.

AdmTERT has the same adenoviral backbone as AdhTERT, but contained a cassette encoding the murine telomerase coding sequence under the control of the CAG promoter.

In vivo vaccination and electroporation: Mice were anesthetized and vaccinated intramuscularly into tibialis with 100 μg of gWiZ™/hTERT, gWiz™/mTERT, or gWizT™ empty plasmid DNA in 50 μL PBS or saline immediately following the injection, a needle was inserted into the tibialis muscle and the muscle was electroporated. The conditions for in vivo electroporation were 180V; 2 pulses/direction=4 pulses total; 20 msec per pulse; needle distance 0.5 cm; 1 sec between pulses.

Stimulation of mouse spleen-derived T cells and CTL-assay in vitro: Spleens were harvested under sterile conditions, and single cell suspensions were prepared. Following lysis of red blood cells, NK-cells were depleted using NK-specific antibodies and magnetic beads. The T cells were then incubated with irradiated stimulator cells in the presence of IL-2. After 5 days, a standard ⁵¹Cr-release assay was performed measuring ⁵¹Cr content in the supernatant of 4 hr cultures of T cells cultured with irradiated ⁵¹Cr-labeled targets. Specific lysis was determined as (experimental lysis minus spontaneous lysis)÷(maximum lysis-spontaneous lysis)×100%.

Cytokine production: Following the 5-day culture, T cells were activated with PMA/lonomycin for 2 h and Brefeldin A for 2 h. After staining the cells for intracellular cytokine expression, the cells were analyzed using a BD-Vantage™ counter.

Results

FIG. 1 shows that three vaccinations with dendritic cells (DCs) primed with an adenovirus hTERT expression vector causes a delay in the growth of B10.2 tumor cells. C57BU6 mice (n=7 in each group) were immunized three times with 1×10⁶ murine bone marrow derived DCs transduced with 200 MOI of AdhTERT, AdmTERT, Ad-Empty or saline, as indicated, at 10-day intervals. 5×10⁵ B10.2 fibrosarcoma cells were injected intradermally in the abdomen area 10 days after the last DC immunization. Tumor growth was monitored twice weekly. Tumor area was calculated as tumor length×width.

B10.2 tumor growth was delayed in mice vaccinated with DC/AdhTERT, DC/Adh/mTERT or DC/AdmTERT plus AdIL-12.

FIG. 2 shows that tumor rejection correlates in this experiment with the presence of CD8⁺ T cells producing IFN-γ. Mice were sacrificed when tumors reached a limit size. Splenocytes, depleted of NK cells, were restimulated with AdhTERT modified B10.2 cells in vitro for 5 days. Then the percentage of CD8⁺/IFN-γ⁺ T cells was determined by flow cytometry after staining with anti-CD8 and anti-IFN-γ.

It was found that the tumor free mice in DC/AdhTERT or DC/Adh/mTERT groups had a higher frequency of CD8⁺/IFN-γ⁺ T cells. Tumor-bearing mice in these two groups as well as mice in control group had low percent of CD8^{+/IFN-γ}⁺ T cells.

FIG. 3 (Upper Panels) show that vaccination with human TERT expression vectors will impart a response that is cross-reactive to epitopes on mouse TERT. C57BU6 mice were immunized eight times intramuscularly at ten-day intervals with 100 μg hTERT or control plasmid DNA (empty), followed by electroporation (180V/0.5 cm/2 pulses). One week after the last vaccination, the spleens were harvested and NK-depleted T cell cultures were stimulated in vitro using AdhTERT modified, IFN-γ treated (10 ng/mL) B16F10 mouse melanoma cells.

After 5 days, a standard cytotoxic T cell mediated target killing ⁵¹Cr release assay (CTL) was performed. The upper panels show CTL activity from individual mice vaccinated with hTERT-DNA, or control (empty plasmid) DNA. Targets were B16F10 mouse melanoma tumor cells transduced with AdhTERT (Left) or unmodified parental line (Right). The results show that immunization with hTERT generates CTLs that are specific for the mouse TERT expressed endogenously by the B16F10 tumor.

FIG. 3 (Lower Panels) show a similar experiment in which mice were vaccinated once with AdhTERT, and 3 times with hTERT-DNA plasmid; or with control vectors. CTLs from the immunized mice lyse B10.2 mouse tumor cells (Left) or C57 mouse fibrosarcoma cells (Right), by virtue of the mouse TERT expressed and presented by these cells. The results show that immunization with human TERT imparts cytotoxic immunity against endogenously expressed antigen expressed by tumor cells of a variety of different tissue types.

FIG. 4 shows IFN-γ expression by T cells from mice vaccinated with hTERT vectors. Data from two experiments with different vaccination regimens are presented. In Panel A, groups of mice were immunized once with AdhTERT, followed by 3 times with gWiz™ hTERT DNA plasmid, or with corresponding control vectors. Spleen cells were then harvested, stimulated in vitro for 5 days, and analyzed by flow cytometry for T cell subtype and IFN-γ expression. The results show specific induction of cytokine-secreting T cell subsets in animals immunized with hTERT, but not control.

In Panel B, individual mice were immunized 8 times with hTERT-DNA plasmid, or with control plasmid plus MPL (adjuvant). The results show that repeated immunization elicits a very high level of IFN-γ expressing T cells, especially those of the CD8 positive (CTL) subset.

FIG. 5 shows that xenogeneic antigen followed by self antigen is better than self antigen alone in generating CTL response against self antigen. C57BU6 mice were immunized intramuscularly four times (at a 10-14 day interval) with 100 μg DNA followed by in vivo electroporation. Group 1 was injected 4 times with AdmTERT. Group 3 was injected two times of hTERT DNA followed by two times of mTERT DNA. Groups 4 and 5 were injected four times with control plasmid or saline respectively. Ten days after the fourth immunization, splenocytes from an animal in each group were harvested. After depletion of NK cells, the cells were stimulated in vitro by culturing with irradiated B16/AdmTERT at a 10:1 ratio for five days. Panel A shows CTL killing of unmodified ⁵¹Cr labeled C57 fibrosarcoma cells. Panel B shows IFN-γ expression in the T cell subsets obtained after culturing in vitro.

The combination human and mouse TERT DNA vaccination was clearly better than mouse TERT DNA alone in generating mTERT-specific CD8 positive CTL response expressing IFN-γ, and capable of lysing mouse tumor cells.

FIG. 6 shows CTL results of a similar experiment in which mice were immunized four times with mTERT DNA, or twice each with hTERT DNA followed by mTERT DNA. Again, the mice immunized with a combination of hTERT and mTERT showed more mTERT-specific CTLs. The level of killing is higher in this experiment because the target cells had been transduced with AdmTERT. This confirms that the killing of the tumor cells is mediated by TERT antigen specific CTLs.

FIG. 7 shows that immunizing with a combination of hTERT and mTERT leads to a specific CTL response against autologous antigen. C57BU6 mice were immunized intramuscularly for total of nine times (at 10-14 day intervals) with 100 μg DNA followed by electroporation, as indicated. Ten days after the ninth immunization, two mice from each group were harvested. Splenocytes (depleted of NK cells) were stimulated in vitro by culturing 5 days with irradiated B16/AdmTERT at 10:1 ratio. Then a standard ⁵¹Cr-release assay was performed to determine mTERT-specific killing (mean±SEM). Target T cells were C57 fibrosarcoma cells transduced with AdmTERT.

The results show that the optimal regiment is a multiple immunization with xenogeneic human TERT, followed by a multiple immunization with autologous mouse TERT.

FIG. 8 shows inhibition of tumor growth in mice immunized with AdhTERT. C57BU6 mice were immunized three times, at an interval of 10-14 days, with AdhTERT virus (1×10⁹ TCID₅₀ per injection) or control virus, either intradermally (ID) or intramuscularly (IM). Two weeks after the last immunization, mice were challenged with B16F10 melanoma cells (2×10⁴/mouse) subcutaneously. Tumor growth was monitored twice weekly, and tumor volume was calculated as (Length×Width×Height)÷2. Significant tumor growth delay was observed in mice receiving AdhTERT vaccination compared with mice receiving control virus. Data in the AdhTERT group (n=14; mean±SEM) are pooled from mice immunized with AdhTERT (IM, n=5), AdhTERT (ID, n=5), AdhTERT plus the adjuvant MPL-SE (ID, n=4). Data in the AdEmpty group (n=12; mean±SE) are pooled from mice treated with AdEmpty (IM, n=4), AdEmpty (ID, n=4), and AdEmpty plus MPL-SE (ID, n=4).

Animals immunized with xenogeneic hTERT (diamonds) resisted tumor growth by almost 3-fold, compared with untreated mice (triangles) or vector control (squares). The results were significant at the level of p<0.1 (AdhTERT vs. PBS); p<0.05 (AdhTERT vs. vector control); and p<0.01 (AdhTERT vs. both controls) using the one tailed Student's t test.

### References

1. Miyake S M, Makimura M, Kanagae Y, et al. Efficient generation of recombinant adenoviruses using adenovirus DNA-terminal protein complex and a cosmid bearing the full-length virus genome. Proc Natl Acad Sci USA 1996; 93:1320-1324.

2. Fu S, Diesseroth A B. Use of cosmid adenoviral vector cloning system for the in vitro construction of recombinant adenoviral vectors. Hum Gene Ther 1997; 8: 1321-13.

3. Steitz J, Bruck J, Steinbrink K, Enk A, Knop J and Tuting T. genetic immunization of mice with human tyrosinase-related protein2: implications for the immunotherapy of melanoma. Int. J cancer 2000, 86: 89-94.

4. Fong L, Brockstedt D, Benike C, Breen J K, Strang G, Ruegg C L, and Engelman E G. Dendritic cell-based xenoantigen vaccination for prostate cancer immunotherapy. J Immunol. 2001, 167: 7150-7156.

5. Hawkins W G, Gold J S, Blachere N E, Bowne W B, Hoos A, lewis J J and Houghton A N. Xenogeneic DNA immunization in melanoma models for minimal residual disease. J Sur. Res., 2001, 102:137-143.

6. Alexander A N, Huelsmeyer M K, Vail D M, Kurzman I D, and MacEwen E G. Phase I/II clinical trial utilizing a tumor cell vaccine encoding xenogeneic Gp100 in canine patients with mestastatic melanoma: immunological and clinical outcome. 2002, Abstract Number 4158, AACR Meeting, San Francisco, Calif.

7. Wolchok J D, Perales M -A, Houghton A N and Bergman P J. Of mice and men (and dogs). 2002, Abstract Number 218, American Society of Gene Therapy Meeting, Boston.

The compositions and procedures described in this disclosure can be effectively modified by routine optimization without departing from the spirit of the invention embodied in the claims that follow.

### Sequence Data

TABLE 4
Sequences Listed in this Disclosure

| SEQ. | Descriptive | |
|---|---|---|
| ID NO: | Annotation | Source |
| 1 | Homo sapiens | GenBank Locus |
| | telomerase reverse | NM 003210. See |
| | transcriptase (TERT) | also Nakamura et |
| | mRNA sequence | al., Science 277:955, |
| | | 1997; and GenBank |
| | | Locus AF015950 |
| 2 | amino acid sequence | |
| 3 | Mus musculus (mouse) | GenBank Locus |
| | TERT cDNA | AF051911 See also |
| | sequence | International |
| | | Patent Publication |
| | | WO 99/27113 and |
| | | GenBank Locus |
| | | NM_009354 |
| 4 | amino acid sequence | |
| 5 | Mesocricetus auratus | GenBank Locus |
| | (golden hamster) | AF149012 |
| | TERT cDNA sequence | |
| 6 | amino acid sequence | |
| 7 | Rattus norvegicus | GenBank Locus |
| | (rat) TERT | AF247818 |
| | cDNA sequence | See also GenBank |
| | | Locus AJ488949 |
| 8 | amino acid sequence | |
| 9 | Canis familiaris (dog) | This invention |
| | TERT genomic sequence | (FIG. 9) |
| 10 | amino acid sequence | This invention |
| | | (FIG. 10) |
| 11 | Consensus TERT | This invention |
| | encoding sequence | (FIG. 9) |
| 12 | Consensus TERT protein | This invention |
| | sequence in which | (FIG. 10) |
| | unspecified positions | |
| | can be any amino acid | |

The invention claimed is:

1. A method for eliciting an immune response in a mammalian subject that is specific for its own telomerase reverse transcriptase (TERT), comprising administering to the subject an immunogenic composition containing a protein with at least 20 consecutive amino acids of TERT of another mammalian species, or a nucleic acid encoding said protein.

2. The method of claim 1, wherein the protein comprises at least 100 consecutive amino acids of TERT of the other mammalian species.

3. The method of claim 1, comprising administering the protein or nucleic acid to the subject on at least four different occasions.

4. The method of claim 1, further comprising subsequently administering a second composition containing a second protein with at least 20 consecutive amino acids of TERT of the same species as the subject, or a nucleic acid encoding said second protein.

5. The method of claim 1, which elicits a cytotoxic T cell response.

6. The method of claim 1, wherein the protein is full-length TERT.

7. The method of claim 1, wherein the immunogenic composition increases telomerase activity in cells surrounding the site of administration.

8. The method of claim 1, wherein the protein lacks telomerase activity when associated with telomerase RNA due to one or more changes in amino acid sequence.

9. The method of claim 1, wherein the composition contains either:
a plurality of different proteins, each comprising at least 20 consecutive amino acids of TERT from one or more mammalian species different from the mammalian subject to which the composition is administered, or
one or more nucleic acids encoding said plurality of proteins.

10. The method of claim 1, wherein the protein comprises at least 20 consecutive amino acids of any of SEQ. ID NOs:4, 6, 8, 10, and 12.

11. The method of claim 1, wherein the composition contains an adenovirus expression vector encoding the protein.

12. The method of claim 1, wherein the composition also contains a factor selected from IL-12, GM-CSF, IL-2 and MPL.

13. An immunogenic composition formulated for human administration, comprising a protein containing at least 20 consecutive amino acids of telomerase reverse transcriptase (TERT) of a non-human mammal, or a nucleic acid encoding said protein, which upon administration to a human patient having a tumor (optionally with simultaneous or sequential administration of another TERT protein or TERT-encoding nucleic acid) elicits an immunological response against human TERT.

14. The composition of claim 13, wherein the protein comprises at least 100 consecutive amino acids of said non-human mammalian TERT.

15. A combination of pharmaceutical compositions formulated for human administration, comprising:
a) a first composition comprising a protein of at least 20 consecutive amino acids of telomerase reverse transcriptase (TERT) of a non-human mammal, or a nucleic acid encoding said protein; and
b) a second composition comprising a second protein of at least 20 consecutive amino acids of human TERT, or a nucleic acid encoding said second protein;
wherein administration of the compositions simultaneously or sequentially to a human patient having a tumor elicits an immunological response against human TERT.

16. The composition of claim 13, wherein the TERT protein of the non-human mammal is full-length TERT.

17. The composition of claim 13, wherein the TERT protein of the non-human mammal has telomerase activity when associated with telomerase RNA component.

18. The composition of claim 13, wherein the TERT protein of the non-human mammal lacks telomerase activity due to one or more changes in amino acid sequence.

19. The composition of claim 13, either containing a plurality of different proteins, each comprising at least 20 consecutive amino acids of TERT from one or more non-human mammals, or containing one or more nucleic acids encoding said plurality of proteins.

20. The composition of claim 13, wherein the TERT protein of the non-human mammal comprises at least 20 consecutive amino acids of any of SEQ. ID NOs:4, 6, 8, 10, and 12.

21. The composition of claim 13, wherein the composition contains an adenovirus expression vector encoding the protein.

22. The composition of claim 13, wherein the composition also contains a factor selected from IL-12, GM-CSF, IL-2, and MPL.

23. The composition(s) of claim 13, packaged with information on its use for eliciting an immunological response against human TERT.

24. The composition(s) of claim 13, packaged with information on its use for treating cancer.


SEQUENCE LISTING (12 sequences)

SEQ ID NO: 1 - DNA, 4015 bp, Homo sapiens
gcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 2 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - DNA, 3426 bp, Mus musculus
gtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatg

SEQ ID NO: 4 - protein, 1122 aa, Mus musculus
MTRAPRCPAVRSLLRSRYREVWPLATFVRRLGPEGRRLVQPGDPKIYRTLVAQCLVCMHWGSQPPPADLSFHQVSSLKELVARVVQRLCERNERNVLAFGFELLNEARGGPPMAFTSSVRSYLPNTVIETLRVSGAWMLLLSRVGDDLLVYLLAHCALYLLVPPSCAYQVCGSPLYQICATTDIWPSVSASYRPTRPVGRNFTNLRFLQQIKSSSRQEAPKPLALPSRGTKRHLSLTSTSVPSAKKARCYPVPRVEEGPHRQVLPTPSGKSWVPSPARSPEVPTAEKDLSSKGKVSDLSLSGSVCCKHKPSSTSLLSPPRQNAFQLRPFIETRHFLYSRGDGQERLNPSFLLSNLQPNLTGARRLVEIIFLGSRPRTSGPLCRTHRLSRRYWQMRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 5 - DNA, 4170 bp, Mesocricetus auratus
gggaggcccggccggatcttgagcgcgatgccccgcgcgccccgttgccgggccgtgcgcgctctgctgcgcagtcaataccgtcaggtgtggccgctggcaaccttcgtgcggcgcctgggacctgagggcaggcagcttgtacaacccggggacccaaaggtcttccgcacgttggtggcccggtgcctagtgtgtgtgccctgggactcacaacctccacctgctgacctttccttccaccaggtgtcatcactgaaggagctggtggccagggtcgtgcagagactctgcgagcgcggcgagaggaacgtgctgacttttggcttcgcgctgcttaacggagcccaaggcggtcctcccatgacattcacaaccagcgtgcgcagctacctgcccaactcggtgactgagtctctgcgcgtcagtggtgcttggatgcttctgctgaaccgagtgggcgacgacttgctggtctacctgctggcccgctgtgcgctttacctgctggtgcccccgagctgtgcctaccaggtgtgcggctcacccctgtaccaaatctgtgccaccgcagaaacctggccttctgtgtcccgcatctacaggcccacacgacccgtgggcagaaattttactcatcttggatccacacaccgggtcaggaacagcagtcaccaggaagcatggaaacccccgcccttgccatctcgagaggcgaagcggagtctaagcatcaccaatagaagtgtgcctccatctaagaaggccaggtgcgatctggccccgagactggagaagggaccctacaggcaggcagttccaaccccatcagacaaaacatgggtgccaaatcctgccaagtcccatgcagtgcctattagtagaactaccaaggaagatttgtcttccggggtgaaggcacctggcctgagtcgctctgggtcagtgtgctataaacacaagcccagttccacatccctgcagtcaccactgtgccaaaatgcctttcagctcagaccatatactgagaccaaacgcttcctctactctagggaaggtggccgagagaggctgaacccctcgttcctactcaacaacctgcagcccagcttgactggggccaggagactggtagagatactctttctaggcatgagacctaggacatcgggaccactctgtgggagacgccgcctatcgaagcgctactggcagatgcggcccctattccagcagttgcttgtgaaccatgcgcggtgcccgtatgtccggctcctcaggtcccattgcaggtttcggaccgcagcccaccaggtggcaggtgccttgaacaccaccagcccacagcgcctcatgaatttgctccgtctacacagcagtccctggcaggtatatggctttcttcaggcctgtgtcggaaagctggtgcctccgggtctctggggttcccggcacaaccagcgacgcttctttaagaacgtgaagcggttcatctccttggggaagtatgacaagctgtcgctgcaggagctgacgtggaagatgaaagttcaagactgcaggtggcttcgcagcagcccagggaacaactgtgtcccggctgcagagcaccgcacgagggaaaggatcctggctgtgttcttgttctggctgatggacgcgtacgtggtagagctgcttcggtcattcttttacgtcacagagaccactttccagaagaaccggctcttcttctaccgaaagagcatgtggagaaggctgcagagcattggagtcaggcatcaccttgagagagtgcggctacaagaactgtctcaagaagaagtcaggcagcgccaggaggcctggccagccatgcccatctgcagactgcgtttcatccccaagcccagtggtcttcggcccattgtgaacatgagttatatgggcaccagagcctttgacaaagggaagcaggctcagcatttcacccagtgtctcaagactctgttcagcgtgctcaactatgaactgacaaaacatactaaccttctgggggcatctgtactgggcctgaatgatatctacaggacctggcggaccttcgtactgcgtgtgcgcactctggacccagcacccaggatgtactttgttaaggcagatgtgacaggggcatatgatgccatcccccaggacaagcttgtggaggttattgccaatatgatcagacacccagacaactcgtactgtatccaccaatatgcagtggtccaaagagatagacaaggccaaatccacaagtccttcaggagacaggtctccaccctctctgacctccagccacacatgggccagttcttgaagcatcttcaggactcagacaccagtgcgctgaggaactccgttgtcattgagcagagcttatctctgaacgaggccagcagcagcctgttcgacttcttcctgcgctttgtgcgtaacagtgtcgtgaagatcggtggcaggtgctatgtccagtgccagggcatcccccagggctccagcctgtccaccctgctctgcagtctgtgtttcggggacatggagaacaagctgtttgctgaagtgcagcaggatgggctgcttttgcgttttgttgatgactttctgttggtgacacctcacctggtccaggcggaagccttcctcagggccctcgtccgtggcatccctgagtacggctgcatgataaacttgcagaagacagtggtaaacttccctgtggacgctggtaccctggatggcacagctccacaccagctgcctgctcactgcctgtttccctggtgtggcttactgctggacactcagactctggaggtgctctgtgactacactggttatgcccggacctcaattaaggccagcctcaccttccagcgcaccttcaaggcggggaggaacatgcgacagaagctcttagctgttttgcggttgaagtgtcacagtctgtttctagacttgcagatgaatagccttcagacagtctgtatcaatgtgtacaagatcttcctgcttcaggcctacaggttccatgcgtgtgcgcttcagcttccctttgaccaacatgttagaaagaaccccgcattctttctgagcatcatctccaacatagcatcctgctgctactccatcctgaaggtcaagaatgcaggaatgacactaaaggccaagggtgcctctggctcatttcctcctgaagctgcacgttggctctgctaccaagccttcctgctcaagctggctggtcattctgtcacctacaagtgtctcctgggacctctcaggacagcacaaaaacagctgtgccggaagctcccaagggcaacaatggccatccttgagactgccgctgacccagccctaagcacagactttcagaccattttggactaacccctccccttcagctggatgaacatgggcattgtagcctcaccactcctggatgcatgccacaagagggactgacctgttgtgaggccagtttgccctccaaaacatgtgccagggctgtagtatgggggttgtccctgtgccgtgtttcctgtggtggacttgatttctttcctgatgcctccggggaagcagctcccacccctctcggtggcagggatccactagccagtaacaccagacagaggagaagatgcctgggcactgggacagtgtgcacggatctgagatgcccggccctgtgtctcttcacatctaacccatggagcctttcccaacggggtcacagtagaaagtgtttttggggggcttacagttttaaaggtttagaatctatgatcatcacggtagggagcatggcagcaaggaggcaggcatgtgtcttctcacgcctaaccgaggagccttttccaatgggccagtggggtcagaatgtctcagagatgaaaacaggactttgaactgatgtgggggtttgaagatgtgtagtccccacagacccgtgtgttcaaatgcttggcctattggaatgtcactattaggaggtgtggcttgttggaggaagtgtgtctgtcactgtgaggacagggctttcaggtctcatatatgctcaaactgtgcccaatacacagacgacttctgctgctatgaataaagatgtagaactctaaaaaaaaaaaaaaaaaaaaaaaaaaa

SEQ ID NO: 6 - protein, 1128 aa, Mesocricetus auratus
MPRAPRCRAVRALLRSQYRQVWPLATFVRRLGPEGRQLVQPGDPKVFRTLVARCLVCVPWDSQPPPADLSFHQVSSLKELVARVVQRLCERGERNVLTFGFALLNGAQGGPPMTFTTSVRSYLPNSVTESLRVSGAWMLLLNRVGDDLLVYLLARCALYLLVPPSCAYQVCGSPLYQICATAETWPSVSRIYRPTRPVGRNFTHLGSTHRVRNSSHQEAWKPPPLPSREAKRSLSITNRSVPPSKKARCDLAPRLEKGPYRQAVPTPSDKTWVPNPAKSHAVPISRTTKEDLSSGVKAPGLSRSGSVCYKHKPSSTSLQSPLCQNAFQLRPYTETKRFLYSREGGRERLNPSFLLNNLQPSLTGARRLVEILFLGMRPRTSGPLCGRRRLSKRYWQMRPLFQQLLVNHARCPYVRLLRSHCRFRTAAHQVAGALNTTSPQRLMNLLRLHSSPWQVYGFLQACVGKLVPPGLWGSRHNQRRFFKNVKRFISLGKYDKLSLQELTWKMKVQDCRWLRSSPGNNCVPAAEHRTRERILAVFLFWLMDAYVVELLRSFFYVTETTFQKNRLFFYRKSMWRRLQSIGVRHHLERVRLQELSQEEVRQRQEAWPAMPICRLRFIPKPSGLRPIVNMSYMGTRAFDKGKQAQHFTQCLKTLFSVLNYELTKHTNLLGASVLGLNDIYRTWRTFVLRVRTLDPAPRMYFVKADVTGAYDAIPQDKLVEVIANMIRHPDNSYCIHQYAVVQRDRQGQIHKSFRRQVSTLSDLQPHMGQFLKHLQDSDTSALRNSVVIEQSLSLNEASSSLFDFFLRFVRNSVVKIGGRCYVQCQGIPQGSSLSTLLCSLCFGDMENKLFAEVQQDGLLLRFVDDFLLVTPHLVQAEAFLRALVRGIPEYGCMINLQKTVVNFPVDAGTLDGTAPHQLPAHCLFPWCGLLLDTQTLEVLCDYTGYARTSIKASLTFQRTFKAGRNMRQKLLAVLRLKCHSLFLDLQMNSLQTVCINVYKIFLLQAYRFHACALQLPFDQHVRKNPAFFLSIISNIASCCYSILKVKNAGMTLKAKGASGSFPPEAARWLCYQAFLLKLAGHSVTYKCLLGPLRTAQKQLCRKLPRATMAILETAADPALSTDFQTILD

SEQ ID NO: 7 - DNA, 1758 bp, Rattus norvegicus
aggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatggc

SEQ ID NO: 8 - protein, 575 aa, Rattus norvegicus
RSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 9 - DNA, 2231 bp, Canis familiaris
atgccgcgagcgccccggtgccgcgccgtgcgcgccctgctgcggggccgctaccgggaggtgctgcccctggccaccttcctgcggcgcctggggcccccgggccggctgctcgtgcggcgcggggacccggcggccttccgcgcgctggtggcgcagtgcctggtgtgcgtgccctggggcgcgcggccgccccccgccgccccgtgcttccgccagctggctttcggcttcgccctgctggacggagcgcgcggcgggccccccgtggccttcacgaccagcgtgcgcagctacctgcccaacacggtaaccgagaccctgcgcggcagcggcgcctgggggctgctgctgcgccgcgtgggcgacgatgtgctcacccacctgctggcgcgctgcgcgctctacctgctggtggctccgagctgcgcctaccaggtgtgcgggccgccgtctacgacctctgcgcccccgcctctctgccgctcccggccccgctccccgctccccgctccccgctcggccggccgggctcgggacctcagacccacacgccaggccagaactcggccagcgcggggcagcccggagcggtcctctggaagcgccagccagtggcggagcagacggcgccacaggccttcccaggccacagctcctgtagcaagccgggtgtacacctgccgggcgcttccccagctggcctgggagggaggccccccggactcgtccaaccaccccagcctggatacatctccggggccccagggagtaccccatgacccagcacaccccgagaccaaacgcttcctctactgctcgggtggcagggagcggctgcgcccctccttcctgctcagtgccctgccgcctaccctgggggcccgcaaactcgtggagaccatctttctgggctctgcgccccagaagccaggggccgcccgcaggatgcgccgcctgcctgcccgctactggcgaatgaggcccctgttccaggagctgcttgggaaccacgcccggtgcccctaccgtgcgctcctcaggacccactgcccgcttcgggccatggccgctaaggaggggtctggcaaccaggcacacaggggagtgggcatctgtcccctggagaggccagtagcagccccccaggagcagacggactccacacgcctggtacagctcctccgacagcacagcagcccctggcaggtgtatgccttcctgagggcctgcctgtgctggctggtgcccactggactctggggctccaggcacaaccagcgccgcttcttgaggaacgtgaagaagttcatctccctgggaaagcacgctaagctctccctgcaggaactgacgtggaagatgaaggtgcgggactgcacctggctgcacgggaacccaggtgaggagtgcagagtgagcaggtgcctggttggcctacaggaaggaccaggctcacagcccgagtgtggtaggcccctccctcccaaccatccatctcggaacaccccttcctctgttgggccggcagcgactgccctgcctgcctctcagccccccgactcccgtcacaaactagtccccatccccagaggctgccgggctgtccacatctgctgccaggagtcatgagacatcacgaaatgagctcttggtggcggccctcatccccttaccccgggcacacatggctcctcataggctgtgcgccacaactcttcaatagtgtgcacctccgagaactgtcagaagcagaggtcaggagacaccgggaagccagacctgctctgctgacctccagactccgcttcctccccaagcctagtgggctgcggccgattgtgaatatggactacatcatgggagccagaacattccacagagacaagaaggtccagcatctcacctcacaactgaagacactgttcagtgtcctgaactatgagcgggcccggcgccccagcctcctaggggcctccatgctgggcatggacgacatccacagggcctggcgcacctttgtgctacgcatacgggcccagaatccggcaccccagctgtactttgtcaaggtggacgtgacgggggcatatgacgccctccctcaggacaggctggtagaggtgattgccaatgtgatcaggcctcaggaaagcacatactgcgtgcgccactatgccgtggtccagaggactgcccggggacacgtccgcaaggccttcaaaagacac

SEQ ID NO: 10 - protein, 743 aa, Canis familiaris
MPRAPRCRAVRALLRGRYREVLPLATFLRRLGPPGRLLVRRGDPAAFRALVAQCLVCVPWGARPPPAAPCFRQLAFGFALLDGARGGPPVAFTTSVRSYLPNTVTETLRGSGAWGLLLRRVGDDVLTHLLARCALYLLVAPSCAYQVCGPPSTTSAPPPLCRSRPRSPLPAPRSAGRARDLRPTRQARTRPARGSPERSSGSASQWRSRRRHRPSQATAPVASRVYTCRALPQLAWEGGPPDSSNHPSLDTSPGPQGVPHDPAHPETKRFLYCSGGRERLRPSFLLSALPPTLGARKLVETIFLGSAPQKPGAARRMRRLPARYWRMRPLFQELLGNHARCPYRALLRTHCPLRAMAAKEGSGNQAHRGVGICPLERPVAAPQEQTDSTRLVQLLRQHSSPWQVYAFLRACLCWLVPTGLWGSRHNQRRFLRNVKKFISLGKHAKLSLQELTWKMKVRDCTWLHGNPGEECRVSRCLVGLQEGPGSQPECGRPLPPNHPSEHPFLCWAGSDCPACLSAPRLPSQTSPHPQRLPGCPHLLPGVMRHHEMSSWWRPSSPYPGHTWLLIGCAPQLFNSVHLRELSEAEVRRHREARPALLTSRLRFLPKPSGLRPIVNMDYIMGARTFHRDKKVQHLTSQLKTLFSVLNYERARRPSLLGASMLGMDDIHRAWRTFVLRIRAQNPAPQLYFVKVDVTGAYDALPQDRLVEVIANVIRPQESTYCVRHYAVVQRTARGHVRKAFKRH

SEQ ID NO: 11 - DNA, 2855 bp, Homo sapiens
atgcccgcgcccccgtgccggccgtgcgccctgctgcgcagcctaccggaggtggccgctggcaccttcgtgcggcgcctggggcccagggcggcgctgtgcaccggggacccgggcttccgccgtggtggcccagtgcctgtgtgcgtgccctgggccacgccccccgccgcctccttccccaggtgtctctgaagagctggtggccggttgcagagctctgcgagcgccggaaacgtgctggctttggcttcgcgctgctacgggcccgggcggccccctggccttcacaccagcgtgcgcagctacctgcccaacacggtacgagcctgcggcaggggctgggctctgctggccggtgggcgacgactgctggtcacctgctggccgctggcgcttactgctggtgcccagctggcctaccaggtgtgcgggcccctgtaccatctggcccccactggccctcgccagcccccgcctgggataacgcgacgagtccaggagcagaaccccggcctgccactcgggcgaggggcggccatgaagtgtgcccaagagccaggcgcccggcggagggcccacaggcaggcccccgcaacggtccaagtcgtccgggtgcctccgaaagtcttggaggctgccgagtcctctcgtggcaacacagccgtccacatcgcgtcaccaccccaagctcacccatacgagaccaaaccttcctctactccgaggtggcgagcggctgcccctcttcctactcagccctgcgccactgactggggccggagactgtggagacatctttctgggctcaggccggacaggcccgcaggacccgcctccgctactggcaatgcggcccctgttccagagctgcttggaaccagccgtgccctaggctcctcaggccatgcgttcgcgcgccacaggtgcggcctgacacccccccgccttgatgctccgcgcacagcagccctggcaggttatggcttctcgggcctgctgcgctggtgcccggctctggggtccaggcacaacagcgccgcttcttagaactaagaagttcatctcctggggaagagcaagcttcctgcaggactgacgtggaagatgaagtcggactgctggctcgcagagcccagggagactgtgtccggctgcagagcaccgcgaggagaggatcctggctgttcctgctggctgatgacctacgtggtagctgctaggtcattcttttacgtcacagagaccacattccagaagaacaggctcttcttctaccgaagagtgtgtggagcaagctgcagagcattggagtcaggcaacaccttgagagagtgcggctacgggagctgtcacaagaagaggtcaggcacaccaggagcctggccagccatgcccatctgcagactgcgcttcatccccaagcccaacggctgcggcccattgtgaacatgagttatacatgggaccagagcttggcagagggaagcaggcccagcatttcacccagcgtctcaagactctgttcagcgtgctcaactatgagcggacaaaacatcctaccttctgggggctctgtactgggcatgaatgacatctacaggacctggcggacctttgtgctgcgtgtgcggctctggacccgcacccaggatgtactttgttaaggcagatgtgacggggctatgatgccatcccccaggacaagctggtggaggttattgccaatatgatcaggcaccggagagcacgtactgtatccgccagtatgcagtggtccagagagatagccaaggccaagtccacaagtccttcaggagacaggtcccaccctctctgacctccagccatacatgggccagttctaagcatctgcaggatcagaccagtgcctgaggaactccgttgtcatcgagcagagctctcttgaatgagcagcagcagcctgttgacttcttcctgccttctgcgtcacagtgtcgtaagatggtgcaggtgctatcagtgccagggcatcccccagggctccagccttccaccctgctctgcagtctgtgtttcgggacatggagaacaagctgtttgctgaggtgcagcgggatgggtgcttttcgttttgttgatgactttctgttggtgaccctcactggccagcaaaaccttcctcagaccctggtcctggcgtcctgagtatggtgcatgataaacttgcagaagacagtggtgaacttccctgtggactggtaccctgggtggcagctccaaccagctgcctgctcactgcctgtttccctggtgtggcttgctgctggacactcagacttggaggtgtctgtgactactcggttatgcccgacctcaattaagcagcctcaccttccagggcttcaagctgggagacatgcgacaagctctcggtcttgcggttgaagtgtcacgtcttttctagacttgcaggtgaacagcctccagacagtctgcatcaatattacaagatcttcctgcttcaggcctacaggttccatgcatgtgtgttcagcttccctttgaccagctgttaggaagaacccacattctttctggcatcatctccacagcatcctgctgctacatcctgaaggtcaagaatcaggaatgacactaaaggcctctggctctttcctcctgaagccgcacattggctctgctaccagccttcctgctcaagctggctgtcattctgtcactacaatgtctcctgggacctctaggacagcccaaaaacgctgtgccggaagctcccagggcgacaatgaccatccttagctgcagctgacccagccctaagcacagactttcagaccattttggactaa

SEQ ID NO: 12 - protein, 1152 aa, Homo sapiens
MPRAPRCRAVRALLRSHYREVLPLATFVRRLGPEGRRLVQPGDPAAFRALVAQCLVCVPWGARPPPAAPSFHQVSSLKELVARVVQRLCERGERNVLAFGFALLDGARGGPPMAFTTSVRSYLPNTVTETLRGSGAWGLLLRRVGDDLLVHLLARCALYLLVAPSCAYQVCGPPLYQIGATTQARPPPHASGRPRRPVGRNFTNLGFCERAWNHSVREAGVPLGLPSPGAKRRGGSASRSLPLPKKARRGAAPEPERTPVGQGSWTPSGRTRVPSDAGSPVVSPARPAEEDLSSKGKVSDLSLSGSVCCKHKPSSPPSLSSPPRPNAFQLRPVYAETKHFLYSSGGRERLRPSFLLSNLQPSLTGARRLVETIFLGSRPWTSGPLCRTHRLSRRYWQMRPLFQELLGNHARCPYVRLLRSHCPLRAAATPVAGALNTSPPQGSVAAPEEVAAPQEQTDSTRLMQLLRQHSSPWQVYGFLRACLCKLVPPGLWGSRHNERRFLKNVKKFISLGKHAKLSLQELTWKMKVRDCAWLRSSPGYESVPAAEHRLRERILAKEHPFLFWLMSVYVVELLRSFFYITESTFQKNRLFFYRKSVWSKLQSIGVRQHLERVRLRELSQEEVRQHQEAWPAMPICRLRFIPKPNGLRPIVNMSYSMGTRAFGRRKQAQHFTQRLKTLFSVLNYERTKHPHLLGASVLGMNDIYRTWRTFVLRVRALDPTPRMYFVKADVTGAYDAIPQDKLVEVIANMIRHSESTYCIRQYAVVQRDAQGQVHKSFRRQVSTLSDLQPYMGQFLKHLQDSDASALRNSVVIEQSISLNEASSSLFDFFLRFLRHSVVKIGGRCYVQCQGIPQGSSLSTLLCSLCFGDMENKLFAEVQRDGLLLRFVDDFLLVTPHLDQAKTFLSTLVRGVPEYGCMINLQKTVVNFPVEPGTLGGTAPYQLPAHCLFPWCGLLLDTQTLEVFCDYSGYARTSIKASLTFQRVFKAGKNMRNKLLSVLRLKCHSLFLDLQVNSLQTVCINIYKIFLLQAYRFHACVIQLPFDQRVRKNPTFFLGIISSQASCCYAILKVKNAGMTLKAKGAAGSFPPEAAHWLCYQAFLLKLAAHSVTYKCLLGPLRTAQKQLCRKLPEATMTILEAAADPALSTDFQTILD